(19) United States
(12) Patent Application Publication
(10) Pub. No.: US 2012/0114584 A1
(43) Pub. Date: May 10, 2012

(54) RELAXER GEL ACTIVATOR

(75) Inventors: Uyi O. Woghiren, University Park, IL (US); Antoinette Fitzpatrick, Bellwood, IL (US); Margaret Stasik, Palos Hills, IL (US)

(73) Assignee: ALBERTO-CULVER COMPANY, Melrose Park, IL (US)

(21) Appl. No.: 13/260,888

(22) PCT Filed: Apr. 13, 2010
(86) PCT No.: PCT/US2010/030905

§ 371 Date: Jan. 18, 2012

Related U.S. Application Data
(60) Provisional application No. 61/169,089, filed on Apr. 14, 2009.

(51) Int. Cl. A61K 8/81; A45D 7/04; A61Q 5/12; A61Q 5/04; A61K 8/92; A61K 8/86

(52) U.S. Cl. 424/70.4; 424/70.2; 424/70.5; 132/203

(57) ABSTRACT

The present invention provides a relaxer gel activator composition, which includes a carbomer and guanidine carbonate as an alkaline neutralizing agent therefor, and a method for producing the relaxer gel activator. In some embodiments, the relaxer gel activator composition includes at least one conditioning agent, which may be PEG-2 dimeadowfoamamidoethylmonium methosulfate. The present invention also provides methods and kits for relaxing hair fibers using the relaxer gel activator of the invention.

### CROSS-REFERENCE TO RELATED APPLICATIONS

This patent application claims the benefit of U.S. Provisional Patent Application No. 61/169,089, filed Apr. 14, 2009, which is incorporated by reference.

### BACKGROUND OF THE INVENTION

Hair relaxing, or lanthionization, is a hair straightening process that is often performed by a professional cosmetologist, e.g., in a salon, or by the subject individual, e.g., at home, with commercially available relaxer kits. Since the treated portion of the hair ultimately moves away from the scalp as the new growth of untreated hair sprouts up from the roots, periodic retreatment may be required in order to maintain a consistent appearance.

The relaxer is applied to the roots of the hair and remains in place for a pre-determined interval (sometimes referred to as a processing period), during which the relaxer alters the hair's texture by a process of controlled damage to the protein structure. The hair can be significantly weakened by the physical overlap of successive applications or by a single excessive one, leading to brittleness, breakage or even alopecia.

Typically, when the relaxer has worked to the desired degree, the hair is rinsed clean. Regardless of the formulation, relaxers typically are alkaline. Accordingly, the hair is often neutralized or even slightly acidified immediately after treatment, e.g., with a suitable shampoo. It is also common to condition the hair promptly after treatment to replace some of the natural oils that are stripped away by the process.

“Lye” relaxers typically utilize sodium hydroxide (also known as NaOH or lye) in combination with water, petroleum jelly, mineral oil and emulsifiers, to create a creamy consistency. On application, the caustic “lye cream” permeates the protein structure of the hair and weakens its internal bonds, causing the natural curls to loosen out (or relax) as the entire fiber swells open. Lye relaxers tend to be rather harsh and caustic, and extremely irritating to the skin and eyes.

Lye relaxers may be labeled as “base” or “no base.” However, the “base” in this context is distinct from the chemical concept of a base, e.g., which is sometimes used synonymously with “alkaline.” Rather, the “base” in this context refers to a preliminary coating, e.g., of petroleum jelly, onto the scalp to protect it from irritation or burning by the lye cream. “No base” creams are considered to be suitable for direct application to the hair roots without a protective “base” layer, although a protective base layer still may be required for individuals who are sensitive to “no base” products.

“No-lye” relaxers generally include three main categories—those which use an alkaline agent other than lye, those which use thioglycolate and those which use a sulfite or bisulfite. No-lye relaxers that use an alkaline agent other than lye operate on the same general principle as lye relaxers but use an alternative alkaline agent, such as potassium hydroxide, lithium hydroxide or guanidine hydroxide.

Due to the instability and poor shelf life of guanidine hydroxide, guanidine hydroxide relaxers are not produced as a pre-formulated product (e.g., in a single container). Rather, guanidine hydroxide relaxers typically exist as two-component systems whereby the relaxer is generated at the time of use by combining a composition that contains a hydroxide compound, e.g., a cream that contains calcium hydroxide (slaked lime), with an “activating solution” (“activator”) that contains guanidine carbonate.

Unlike guanidine hydroxide, guanidine carbonate is relatively stable and therefore is amenable to long-term storage. The guanidine carbonate serves as a guanidine hydroxide precursor. Thus, when the guanidine carbonate composition (“activator”) and the composition containing the hydroxide compound are mixed, the hydroxide compound reacts with the guanidine carbonate to produce guanidine hydroxide, the active agent used for relaxing the hair. The composition that contains the hydroxide compound is sometimes referred to as the “base” component (e.g., when the hydroxide-containing composition is a cream, the component may be referred to as a “cream base”). The “base” in this context is entirely distinct from “base” in the chemical context (synonymous with alkaline) and also is distinguished from “base” in the context of “base” vs. “no base” categories for lye relaxers.

While guanidine hydroxide relaxers tend to be slightly less alkaline than “lye” relaxers, guanidine hydroxide relaxers are still highly alkaline and suffer from the same drawbacks associated with relaxers in general, e.g., hair brittleness and hair breakage. In addition, the guanidine carbonate activator component used in conventional guanidine hydroxide relaxers are free-flowing liquids, which creates handling difficulties and raises potential hazards. In particular, the guanidine carbonate itself is highly irritating to the skin and eyes, and great care must be taken to avoid splashing, spilling or running of the solution during mixing and application. Conventional guanidine hydroxide relaxers also are difficult to use because the composition tends to form clumps and produce a non-uniform mixture when the activator and hydroxide compositions are mixed.

There is thus a need for a guanidine hydroxide relaxer that is easier and safer to handle, prepare and apply. There is also a need for a guanidine hydroxide relaxer that results in less brittleness and less breakage of the hair. The present invention provides such a relaxer. These and other advantages of the invention, as well as additional inventive features, will be apparent from the description of the invention provided herein.

### BRIEF SUMMARY OF THE INVENTION

The present inventors have unexpectedly found that combining a carbomer with guanidine carbonate produces an organic gel that is not only stable and highly effective as an activator for use in hair relaxer systems, but also mixes far more effectively with conventional hydroxide creams to produce a product with consistency and uniformity that is superior to relaxers produced from conventional activators. The relaxer gel activator of the invention is also safer and easier to handle than conventional relaxer activator solutions, and significantly reduces the hazards of splashing, spilling and running normally associated with conventional relaxers. The present inventors have further discovered that the gel activator of the invention is compatible with conditioning agents, and that incorporating a conditioning agent (e.g., a quaternary ammonium conditioner) in the gel activator unexpectedly produces a hair relaxer that not only meets or exceeds the performance of known commercial products, e.g., in terms of breakdown of natural curl, but also results in a significant reduction in hair breakage. In addition, the relaxer gel activator of the invention avoids the need for alkaline neutralizing agents that are conventionally used for carbomer-based gels (e.g., triethanolamine, sodium hydroxide and aminomethyl propanol).

The present invention thus provides a relaxer activator in the form of a gel, which includes a carbomer and, as a neutralizing agent, guanidine carbonate. Combining the relaxer gel activator of the invention with a suitable hydroxide composition (e.g., a calcium hydroxide cream base) produces a guanidine hydroxide relaxer, which can be applied, e.g., at home or in a salon setting, for relaxing (e.g., straightening) hair using well-known techniques. The gel activator of the invention generally includes a carbomer, an alkaline neutralizing agent consisting essentially of guanidine carbonate and a fluid medium (e.g., an aqueous fluid medium). The carbomer is present in an amount effective to form a gel in the fluid medium, and the guanidine carbonate is present in an amount effective to neutralize the carbomer sufficiently to produce a gel in the fluid medium.

The present invention also provides a method for producing the relaxer gel activator of the invention. The method includes neutralizing a carbomer with guanidine carbonate in a fluid medium (e.g., an aqueous medium) to produce the gel activator composition.

The present invention additionally provides a method for relaxing (e.g., straightening) hair fibers. The method includes combining the gel activator composition of the invention with a hydroxide composition, to produce guanidine hydroxide, and contacting the hair with the guanidine hydroxide to relax the hair fibers.

The present invention further provides a kit that includes a first container with the relaxer gel activator composition of the invention contained therein, and a second container with a hydroxide composition contained therein. The kit preferably includes instructions for reacting the gel activator and hydroxide compositions to produce guanidine hydroxide, and contacting hair with the guanidine hydroxide to relax the hair.

The hydroxide composition generally includes a suitable carrier (e.g., a cream base) and a hydroxide compound (e.g., a metal hydroxide, e.g., calcium hydroxide). The hydroxide compound is present in an amount effective to convert at least a portion of the guanidine carbonate into guanidine hydroxide. “Base” compositions that are conventionally used as the hydroxide ion source in commercial guanidine hydroxide relaxers (e.g., conventional calcium hydroxide cream “bases”) may be used as the hydroxide composition in the relaxing method and kit of the present invention.

The relaxer gel activator of the invention does not require any alkaline neutralizing agents other than guanidine carbonate, and thus avoids the need for alkaline neutralizing agents that are conventionally used for carbomer-based gels. Accordingly, it is preferred that the gel activator of the invention is substantially free of any alkaline neutralizing agent other than guanidine carbonate. Preferably, the gel activator of the invention contains less than about 2 wt. %, less than about 1 wt. %, e.g., less than about 0.5 wt. %, e.g., less than about 0.2 wt. %, e.g., less than about 0.1 wt. %, e.g., less than about 0.01 wt. %, or, e.g., less than about 0.001 wt. %, of an alkaline neutralizing agent other than guanidine carbonate, or no other alkaline neutralizing agent at all.

### BRIEF DESCRIPTION OF THE DRAWINGS

FIG. 1A illustrates the appearance and uniformity of a guanidine hydroxide relaxer composition prepared from a conventional activator.

FIG. 1B illustrates the appearance and uniformity of a guanidine hydroxide relaxer composition prepared from the relaxer gel activator of the present invention.

### DETAILED DESCRIPTION OF THE INVENTION

The present invention is predicated, at least in part, on the surprising and unexpected discovery that combining a carbomer with guanidine carbonate produces an organic gel that is not only stable and highly effective as an activator for hair relaxer systems, but also exhibits superior mixing and handling properties when used as the activator in a relaxer system. The gel activator of the invention mixes easily with conventional hydroxide creams and produces a relaxer with a consistency and uniformity that is superior to relaxers produced from conventional activators. The gel activator of the invention also has a consistency that significantly reduces the likelihood of splashing or spilling of the guanidine carbonate (a known irritant) onto exposed skin or into the eyes. The relaxer gel of the invention is also advantageous in that additional neutralizing agents are not required. Accordingly, the relaxer gel of the invention avoids the need for alkaline neutralizing agents that are conventionally employed in carbomer-based gels.

Any suitable carbomer can be used as the carbomer in the relaxer gel activator of the invention. Suitable carbomers include polyacrylic acid polymers such as, e.g., polyacrylic acid polymers sold under the trademarks Carbopol® and Synthalen®. Any suitable quantity of carbomer can be used in the relaxer gel activator of the invention. The relaxer gel activator of the invention desirably includes a carbomer in an amount effective to produce a stable gel in the fluid medium. Preferably, the relaxer gel activator of the invention includes a carbomer in an amount of from about 0.5 wt. % to about 10 wt. %, e.g., from about 0.5 wt. % to about 7 wt. %, e.g., from about 0.5 wt. % to about 5 wt. %, e.g., from about 1 wt. % to about 7 wt. %, from about 1 wt. % to about 5 wt. %, e.g., from about 1 wt. % to about 4 wt. %, e.g., from about 1 wt. % to about 3.5 wt. %, e.g., from about 1 wt. % to about 3 wt. %, e.g., from about 1.5 wt. % to about 7 wt. %, e.g., from about 1.5 wt. % to about 5 wt. %, e.g., from about 1.5 wt. % to about 4 wt. %, e.g., from about 1.5 wt. % to about 3.5 wt. %, e.g., from about 1.5 wt. % to about 3 wt. % or, e.g., about 1.5%, based on the total weight of the relaxer gel activator composition.

Any suitable quantity of guanidine carbonate can be used in the relaxer gel activator composition of the invention. The relaxer gel activator of the invention desirably includes guanidine carbonate in an amount effective to neutralize the carbomer sufficiently to produce a gel in the fluid medium. Preferably, the relaxer gel activator of the invention includes guanidine carbonate in an amount of from about 10 wt. % to about 50 wt. %, e.g., from about 10 wt. % to about 45 wt. %, e.g., from about 10 wt. % to about 40 wt. %, e.g., from about 10 wt. % to about 35 wt. %, from about 10 wt. % to about 30 wt. %, e.g., from about 10 wt. % to about 25 wt. %, e.g., from about 15 wt. % to about 50 wt. %, e.g., from about 15 wt. % to about 45 wt. %, e.g., from about 15 wt. % to about 40 wt. %, e.g., from about 15 wt. % to about 35 wt. %, e.g., from about 15 wt. % to about 30 wt. %, e.g., from about 15 wt. % to about 25 wt. %, e.g., from about 20 wt. % to about 50 wt. %, e.g., from about 20 wt. % to about 45 wt. %, e.g., from about 20 wt. % to about 40 wt. %, e.g., from about 20 wt. % to about 35 wt. %, e.g., from about 20 wt. % to about 30 wt. %, e.g., from about 25 wt. % to about 50 wt. %, e.g., from about 25 wt. % to about 45 wt. %, e.g., from about 25 wt. % to about 40 wt. %, e.g., from about 25 wt. % to about 35 wt. %, or, e.g., from about 25 wt. % to about 30 wt. %, e.g., about 28 wt. %, e.g., 28 wt. %, e.g., 28.2 wt. %, based on the total weight of the relaxer gel activator composition.

The relaxer gel activator of the invention also may include a conditioning agent, if desired. When included, the conditioner preferably is used in an amount effective to impart conditioning properties to the hair, e.g., ease of combing, smoothness and after-feel, when used in a relaxer system. Cationic conditioners, e.g., quaternary ammonium conditioners, are among the preferred conditioning agents. Meadowquat® conditioners (marketed by Fancor® Ltd.) such as, e.g., Meadowquat® HG and Meadowquat® HG-70, are particularly preferred.

The present inventors have surprisingly discovered that including a Meadowquat® conditioner, e.g., PEG-2 dimeadowfoamamidoethylmonium methosulfate, in the relaxer gel activator of the invention can produce a guanidine hydroxide relaxer, which exhibits a 30-60% reduction in hair breakage (following relaxing/lanthionization) relative to guanidine hydroxide relaxers prepared from conventional activators.

When included in the relaxer gel activator of the invention, a Meadowquat® conditioner preferably is used in an amount effective to impart conditioning properties to the hair, e.g., ease of combing, smoothness and after-feel, and more preferably in an amount effective to impart reduced breakage, when used in a relaxer system. Meadowquat® conditioners are known in the art and typically include as the conditioning agent quaternary ammonium compounds derived from one or more meadowfoam seed oil components. Any suitable Meadowquat® conditioner may be used. Especially preferred Meadowquat® conditioners include PEG-2 dimeadowfoamamidoethylmonium methosulfate derivatives of the formula:

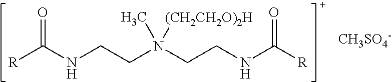

where R is a long-chain (e.g., C₂₀, C₂₂) unsaturated fatty alkyl found in fatty acids derived from meadowfoam seed oil. Meadowquat® may contain hexylene glycol as a solvent or co-solvent. When a conditioner such as, e.g., a Meadowquat® conditioner, is used in the relaxer gel activator of the present invention, the relaxer gel activator preferably includes from about 0.0001 wt. % to about 10 wt. %, e.g., from about 0.001 wt. % to about 10 wt. %, e.g., from about 0.01 wt. % to about 10 wt. %, e.g., from about 0.1 wt. % to about 10 wt. %, e.g., from about 0.5 wt. % to about 10 wt. %, e.g., from about 0.5 wt. % to about 9 wt. %, e.g., from about 0.5 wt. % to about 8 wt. %, e.g., from about 0.5 wt. % to about 7 wt. %, e.g., from about 0.5 wt. % to about 6 wt. %, e.g., from about 0.5 wt. % to about 5 wt. %, e.g., from about 0.5 wt. % to about 4 wt. %, e.g., from about 0.5 wt. % to about 3 wt. %, e.g., from about 0.5 wt. % to about 2 wt. %, e.g., from about 0.5 wt. % to about 1 wt. %, e.g., from about 1 wt. % to about 10 wt. %, e.g., from about 1 wt. % to about 9 wt. %, e.g., from about 1 wt. % to about 8 wt. %, e.g., from about 1 wt. % to about 7 wt. %, e.g., from about 1 wt. % to about 6 wt. %, e.g., from about 1 wt. % to about 5 wt. %, e.g., from about 1 wt. % to about 4 wt. %, e.g., from about 1 wt. % to about 3 wt. %, e.g., from about 1 wt. % to about 2 wt. %, e.g., from about 2 wt. % to about 10 wt. %, e.g., from about 2 wt. % to about 9 wt. %, e.g., from about 2 wt. % to about 8 wt. %, e.g., from about 2 wt. % to about 7 wt. %, e.g., from about 2 wt. % to about 6 wt. %, e.g., from about 2 wt. % to about 5 wt. %, e.g., from about 2 wt. % to about 4.5 wt. %, e.g., from about 2 wt. % to about 4 wt. %, e.g., from about 2 wt. % to about 3.5 wt. %, e.g., from about 2.5 wt. % to about 4 wt. %, e.g., from about 2.5 wt. % to about 3.5 wt. %, or, e.g., from about 2 wt. % to about 3 wt. %, of the conditioner, based on the total weight of the relaxer gel activator composition.

The fluid medium of the relaxer gel activator of the invention preferably is an aqueous fluid medium, e.g., water or an aqueous mixture of water in combination with one or more co-solvents and/or other suitable liquid media. Any suitable amount of water can be used in the relaxer gel activator composition. Preferably, the relaxer gel activator composition includes from about 50 wt. % to about 90 wt. %, e.g., from about 50 wt. % to about 85 wt. %, e.g., from about 50 wt. % to about 80 wt. %, e.g., from about 50 wt. % to about 75 wt. %, e.g., from about 50 wt. % to about 70 wt. %, e.g., from about 60 wt. % to about 90 wt. %, e.g., from about 60 wt. % to about 85 wt. %, e.g., from about 60 wt. % to about 80 wt. %, e.g., from about 60 wt. % to about 75 wt. %, e.g., from about 60 wt. % to about 70 wt. %, or, e.g., from about 64 wt. % to about 67 wt. %, e.g., about 65 wt. %, of water, based on the total weight of the relaxer gel activator composition.

Although not essential, an effective amount of at least one preservative may be incorporated in the relaxer gel activator of the invention. For example, the preservative can be selected to kill bacteria that might otherwise be sustained or multiply in the composition. Such preservatives are well known to those of ordinary skill in the art, and examples of suitable preservatives will thus be readily apparent to those of ordinary skill in the art.

Examples of suitable preservatives include benzyl benzoate, disodium EDTA, trisodium EDTA, tetrasodium EDTA, isothiazolinones (e.g., KATHON CG, commercially available from Rohm & Haas), DMDM hydantoin, and combinations thereof. DMDM hydantoin, also referred to as 1,3-dimethylol-5,5-dimethyl hydantoin, is commercially available from Lonza, Inc. of Fairlawn, N.J. under the trademark GLYDANT®. In one embodiment, the preservative includes disodium EDTA and DMDM hydantoin in combination. The preservative may be included, e.g., in an amount of from about 0.02 wt. % to about 1 wt. %, e.g., from about 0.1 wt. % to about 1 wt. %, e.g., from about 0.1 wt. % to about 0.5 wt. %, or, e.g., from about 0.2 wt. % to about 0.3 wt. %, based on the total weight of the relaxer gel activator composition.

The relaxer gel activator composition of the present invention may also include, optionally, a thickener in addition to carbomer, if desired, provided that the additional thickener is sufficiently compatible.

Other optional ingredients that may be useful in the relaxer gel activator of the invention include, for example, humectants, aloe vera gel, hexylene glycol, opacifiers, hair strengtheners, fragrances, dyes, or a combination thereof. Suitable humectants may include, e.g., glycerin, propylene glycol, or a combination thereof. One or more opacifiers optionally may be included, e.g., to modify the appearance of the relaxer gel activator composition, if desired.

The relaxer gel activator composition of the invention can have any suitable pH. The relaxer gel activator composition of the invention preferably has an alkaline pH, e.g., from about pH 9 to about pH 11, e.g., from about pH 9.9 to about pH 10.2.

The relaxer gel activator of the invention preferably has a viscosity of from about 20,000 cps to about 60,000 cps, e.g., from about 20,000 cps to about 50,000 cps, e.g., from about 20,000 cps to about 40,000 cps, e.g., from about 20,000 cps to about 30,000 cps, e.g., from about 30,000 cps to about 60,000 cps, e.g., from about 30,000 cps to about 50,000 cps, e.g., from about 30,000 cps to about 40,000 cps, e.g., from about 40,000 cps to about 60,000 cps, e.g., from about 40,000 cps to about 50,000 cps, or, e.g., from about 50,000 cps to about 60,000 cps, when measured by Brookfield Viscometer, RVT, spindle “C,” speed “5” for 60 seconds at 25° C.

The present invention also provides a method for producing the relaxer gel activator of the invention. The method generally includes neutralizing a carbomer with guanidine carbonate in a fluid medium to produce the gel composition. Preferably, no other alkaline neutralizing agent other than guanidine carbonate is used. Methods for producing the relaxer gel activator of the invention are described in more detail herein.

The present invention further provides a method for relaxing (e.g., straightening) hair fibers. The method includes combining the gel activator of the invention with a hydroxide composition, to form guanidine hydroxide, and contacting the hair fibers with the guanidine hydroxide to relax the hair fibers. The hydroxide composition generally includes a carrier (e.g., a cream base) and a hydroxide compound (e.g., a metal hydroxide, e.g., calcium hydroxide). The hydroxide compound is present in an amount effective to convert at least a portion of the guanidine carbonate into guanidine hydroxide. In one aspect, the method includes combining the gel activator of the invention with the hydroxide composition directly on the hair fibers, forming guanidine hydroxide directly on the hair. In another aspect, the method includes reacting the gel activator of the invention with the hydroxide composition (e.g., by mixing the components together in a container) to produce a relaxer containing guanidine hydroxide, and thereafter contacting hair with the relaxer containing the guanidine hydroxide to relax the hair fibers.

The hydroxide compound used in the hydroxide composition can include any suitable compound that contains hydroxide ion and is capable of converting guanidine carbonate into guanidine hydroxide. Suitable hydroxide compounds can include, e.g., sodium hydroxide, calcium hydroxide, lithium hydroxide and/or potassium hydroxide. In one aspect, the hydroxide compound used in the hydroxide composition is calcium hydroxide. In some instances, the use of calcium hydroxide may be advantageous in that, upon reaction with guanidine carbonate, a water-insoluble by-product (calcium carbonate) is formed, which precipitates out of solution and favors completion of the reaction. Chelating agents also may be optionally used, e.g., in the relaxer gel activator, to help drive the reaction toward completion. Suitable chelating agents may include, e.g., EDTA (which may also function as a preservative), or chelating polymers, e.g., polymers that contain one or more chelating functional groups.

Suitable amounts of hydroxide in the hydroxide composition generally include amounts that are sufficient to produce a hair relaxing effective amount of guanidine hydroxide when the relaxer gel is mixed with the hydroxide composition. Suitable amounts of hydroxide can include, e.g., from about 0.1 mol/kg to about 1.5 mol/kg, from about 0.3 mol/kg to about 1.5 mol/kg, from about 0.3 mol/kg to about 1.1 mol/kg, from about 0.3 mol/kg to about 1 mol/kg, from about 0.3 mol/kg to about 0.9 mol/kg, from about 0.3 mol/kg to about 0.8 mol/kg, from about 0.3 mol/kg to about 0.7 mol/kg, from about 0.4 mol/kg to about 1.3 mol/kg, from about 0.4 mol/kg to about 1.2 mol/kg, from about 0.4 mol/kg to about 1.1 mol/kg, from about 0.4 mol/kg to about 1 mol/kg, from about 0.4 mol/kg to about 0.9 mol/kg, from about 0.4 mol/kg to about 0.8 mol/kg, from about 0.4 mol/kg to about 0.7 mol/kg, from about 0.5 mol/kg to about 1.3 mol/kg, from about 0.5 mol/kg to about 1.2 mol/kg, from about 0.5 mol/kg to about 1.1 mol/kg, from about 0.5 mol/kg to about 1 mol/kg, from about 0.5 mol/kg to about 0.9 mol/kg, from about 0.5 mol/kg to about 0.8 mol/kg, from about 0.5 mol/kg to about 0.7 mol/kg, from about 0.6 mol/kg to about 1.5 mol/kg, from about 0.6 mol/kg to about 1.3 mol/kg, from about 0.6 mol/kg to about 1.1 mol/kg, from about 0.6 mol/kg to about 1 mol/kg, from about 0.6 mol/kg to about 0.9 mol/kg, from about 0.6 mol/kg to about 0.8 mol/kg, from about 0.6 mol/kg to about 0.7 mol/kg, from about 0.7 mol/kg to about 1.5 mol/kg, from about 0.7 mol/kg to about 1.3 mol/kg, from about 0.7 mol/kg to about 1.1 mol/kg, from about 0.7 mol/kg to about 1 mol/kg, from about 0.7 mol/kg to about 0.9 mol/kg, from about 0.7 mol/kg to about 0.8 mol/kg, from about 0.8 mol/kg to about 1.5 mol/kg, from about 0.8 mol/kg to about 1.3 mol/kg, from about 0.8 mol/kg to about 1.1 mol/kg, from about 0.8 mol/kg to about 1 mol/kg, from about 0.8 mol/kg to about 0.9 mol/kg, from about 0.9 mol/kg to about 1.5 mol/kg, from about 0.9 mol/kg to about 1.3 mol/kg, from about 0.9 mol/kg to about 1.1 mol/kg, from about 0.9 mol/kg to about 1 mol/kg, from about 1 mol/kg to about 1.5 mol/kg, from about 1 mol/kg to about 1.3 mol/kg, or, from about 1 mol/kg to about 1.1 mol/kg, of hydroxide. In some embodiments, the amount of hydroxide in the hydroxide composition is from about 0.65 mol/kg to about 0.95 mol/kg (e.g., from about 4.8 wt. % to about 7 wt. % calcium hydroxide), e.g., about 0.81 mol/kg calcium hydroxide (e.g., about 6 wt. % calcium hydroxide).

The hydroxide composition may include suitable additives, e.g., additives that are conventionally used in hydroxide “base” compositions. Such suitable additives can include, e.g., one or more solvents (e.g., water), petrolatum, one or more oils (e.g., mineral oil), one or more fatty alcohols (e.g., cetyl alcohol, stearyl alcohol), one or more polyoxyalkylene-fatty ethers (e.g., ceteth-20, steareth-20), plant extracts (e.g., aloe, proteins, fragrances, etc.), and the like, and combinations thereof.

In one aspect, the hydroxide composition is in the form of a cream. Cream “base” compositions that are conventionally used as the hydroxide ion source in commercial guanidine hydroxide relaxers (e.g., conventional calcium hydroxide cream “bases”) may be used as the hydroxide composition in accordance with the present invention. Formulating such compositions is well within the skill of the ordinarily skilled artisan.

The relaxer gel activator of the present invention may be combined with the hydroxide composition (base component) in any suitable ratio to produce a composition containing guanidine hydroxide in an amount effective to relax hair. One of ordinary skill in the art will recognize that suitable mixing ratios will depend, at least in part, on the concentration of guanidine in the relaxer gel activator and the concentration of hydroxide in the base component. Suitable guanidine concentrations can be achieved, e.g., by formulating the relaxer gel activator of the invention with guanidine carbonate at concentrations used in conventional liquid guanidine carbonate relaxer activators. Suitable hydroxide concentrations can be achieved, e.g., by formulating the hydroxide component with a hydroxide compound (e.g., calcium hydroxide) at concentrations used in conventional relaxer base component formulations. The determination of a suitable ratio for mixing the guanidine relaxer gel activator of the invention and base component is well within the skill of the ordinarily skilled artisan.

One of ordinary skill in the art also will recognize that, in some cases, it may be desirable to use a mole excess of hydroxide relative to guanidine carbonate. For instance, when the hydroxide compound in the base component is calcium hydroxide, it may be desirable to combine the relaxer gel activator and base component in such a ratio as to achieve a calcium hydroxide/guanidine carbonate mole ratio of, e.g., from about 1.2 to about 2.5, from about 1.3 to about 2.5, from about 1.4 to about 2.5, from about 1.5 to about 2.5, from about 1.5 to about 2.4, from about 1.5 to about 2.3, from about 1.5 to about 2.2, from about 1.5 to about 2.1, from about 1.5 to about 2, from about 1.6 to about 2, from about 1.7 to about 2, from about 1.8 to about 2, or from about 1.8 to about 1.9, or from about 2.1 to about 2.2 (moles of calcium hydroxide to moles of guanidine carbonate).

It may be convenient to formulate the relaxer gel activator of the invention and base component using concentrations of guanidine carbonate and hydroxide, respectively, such that a desired hydroxide/guanidine mole ratio and an effective concentration of guanidine hydroxide can be achieved by combining roughly equal volumes (i.e., about 1:1 v/v). Formulating the relaxer gel activator and base components to achieve such a result is well within the skill of the ordinarily skilled artisan. Suitable of relaxer gel activator/hydroxide component mixing ratios also may include mixing ratios of, e.g., about 1:2 v/v, about 1:3 v/v, about 1:4 v/v, about 1:5 v/v, about 2:1 v/v, about 3:1 v/v, about 4:1 v/v, or about 5:1 v/v.

Once the relaxer gel activator of the invention is combined with a suitable hydroxide composition, the resulting relaxer containing the guanidine hydroxide can be applied (e.g., at home or in a salon) using techniques that are well known in the art for relaxing hair. The resulting relaxer composition, which contains guanidine hydroxide, can be applied to the hair and allowed to remain in contact with the hair for a time that is sufficient to relax the hair and achieve a desired effect. After removal of the relaxer composition from the hair, e.g., after rinsing the relaxer composition from the hair, the hair may be styled using a hair dryer, a heating iron, etc.

The present invention further provides a kit that includes a first container containing the relaxer gel activator of the invention, and a second container containing a hydroxide composition as described herein. The kit preferably includes instructions for reacting the gel activator and hydroxide compositions to produce guanidine hydroxide, and contacting hair with the guanidine hydroxide to relax the hair. The instructions may describe one or more of the methods for relaxing (e.g., straightening) hair as described herein. The kit thus may include all of the components necessary for relaxing hair in accordance with the present invention, and may further include suitable pre-treatment and/or post-treatment compositions. Suitable pre-treatment compositions can include, for example, compositions containing deionized water, hydroxyethylcellulose, glycerin, methylparaben, disodium EDTA, cocamide MEA, polyquaternium-22 (40%), PEG-12 dimethicone, cetearyl alcohol, sorbitan oleate, ceteareth-20, oleth-5, oleth-20, coconut oil, phenoxyethanol, hydroxypropyltrimonium honey, hydroxypropyl bis-stearyldimonium chloride, Salcare® SC-92, shea butter, aloe vera, and fragrance, and suitable pre-treatment compositions that are otherwise known in the art.

One or more steps may be included in the post-treatment of the hair. Hair treated with a relaxer composition produced according to the present invention may be rinsed following application and straightening. The hair may also be shampooed and/or conditioned following application and straightening. The shampoo may contain components to neutralize the alkaline environment that may exist following application and straightening. All post-relaxation treatment shampooing and/or conditioning may be performed in one or multiple applications. For example, after relaxation, the hair may be rinsed, subjected to one or more shampoo treatments, and then subjected to one or more conditioning treatments. The shampoo and conditioning treatments may be done successively (e.g., two shampoo treatments followed by two conditioning treatments) or staggered (e.g., one or more cycles of shampooing followed by conditioning). Suitable post-treatment compositions can include, for example, compositions containing quaternium-80, propylene glycol, dimethicone PEG-7 isostearate, polyquaternium-32, mineral oil, PPG-1 trideceth-6, water, DMDM hydantoin, dimethicone, mineral oil, glycerin, deionized water, panthenol, shea butter, fragrance, coconut oil, honey, and wheat proteins and as otherwise described herein and post-treatment compositions that are otherwise known in the art.

The following examples further illustrate the invention but, of course, should not be construed as in any way limiting its scope.

### Example 1

This example demonstrates methods for producing exemplary relaxer gel activator compositions.

The lists of ingredients and the weight percentages for each of the Activators 1A-1J are summarized in Tables 1A-1C.

The same general procedure is used to prepare Activators 1A-1H. Add the deionized water to a beaker. Slowly sprinkle the carbomer into the water and mix until well hydrated. Add the guanidine carbonate and mix until a smooth and homogeneous gel is formed. Individually add the glycerin, the red #33 (CI 17200), and, if present, the aloe vera gel, Meadowquat® HG-70 (PEG-2 dimeadowfoamamidoethylmonium methosulfate (70% active), from Fanning Corp., Chicago, Ill.), African hydration complex (a light yellow liquid oil containing carthamus tinctorius (safflower) seed oil (50%), crambe abyssinica seed oil (30%), butyrospermum parkii (shea butter) (5%), olea europaea (olive) oil unsaponifiables (10%), and lecithin (5%)), DC (Dow Corning) silicone, safflower seed oil, and/or sunflower seed oil, mixing well after each addition.

Activator 1I and 1J are prepared as follows. Add deionized water (65.195121% of the final wt. % for Activator 1I, 64.995441% of the final wt. % for Activator 1J) into a stainless steel main compounding tank. Charge the water cold (60-78° F./16-26° C.) where available. Start mixing with a turbine mixer (vigorous counterclockwise direction) and a side sweep mixer (moderate forward direction). Slowly add the carbomer by sprinkling the powder with vigorous mixing. Avoid the creation of any lumps. If compounding the carbomer is problematic, before addition of the carbomer, 0.04% (based on total final batch weight) of hydrochloric acid can be added. The pH of the water mix desirably should be lower than 2.45 in order for the carbomer to disperse easily. The HCl can be added in increments of 0.001% of the total final batch weight, if necessary, in order to achieve a pH of 2.45. After addition of the carbomer, mix the mixture for a minimum of 2 hours or until all the carbomer is in solution. Take one or more samples from the mix vessel to verify that all of the carbomer is dispersed with no lumps or particulates. Visually verify that the tank is free from undispersed carbomer. Increase the mixing as necessary to disperse any remaining particulates, and resample the mixture periodically until the batch is free from particulates. With vigorous mixing, add the guanidine carbonate powder. Mix the mixture for a minimum of 60 minutes with the turbine mixer at a moderate to vigorous speed in a counterclockwise direction and the side sweep mixer at a moderate speed in a forward direction. Take samples to verify that all particles are dissolved. With moderate mixing, add the glycerin. Add the aloe barbadensis leaf juice and Meadowquat HG-70. Make a premix of the red 33. Add the red 33 separately to water (0.104479% of the final wt. % for Activator 1I and 0.104159% of the final wt. % for Activator 1J), with the water between 120-140° F., where available, to improve dissolution of the dye. Mix the dye premix well until no particles remain in the premix, and then add the premix to the main batch tank, with moderate agitation, when the dye is completely dissolved. Mix the main tank for a minimum of 60 minutes (turbine mixer at a moderate counterclockwise direction, side sweep mixer at a moderate forward direction). Sample the batch and discontinue mixing and cooling. Filter the mixture using an air diaphragm and positive displacement pump and 80 mesh filter. Fill the mixture into containers at room temperature.

TABLE 1A

| Activator | 1A | 1B | 1C | 1D |
|---|---|---|---|---|
| Composition | (wt. %) | (wt. %) | (wt. %) | (wt. %) |
| Deionized water | 66.7995 | 65.2996 | 66.7996 | 64.7996 |
| Carbomer | 3 | 1.5 | 3 | 2 |
| Guanidine carbonate | 28 | 28 | 28 | 28 |
| Glycerin | 2 | 2 | 2 | 2 |
| Red #33 | 0.0004 | 0.0004 | 0.0004 | 0.0004 |
| Aloe vera gel | 0.2 | 0.2 | 0.2 | 0.2 |
| Meadowquat ® HG-70 | 0.0001 | 3 | 0 | 0 |
| DC silicone | 0 | 0 | 0 | 0 |
| Safflower seed oil | 0 | 0 | 0 | 0 |
| Sunflower oil | 0 | 0 | 0 | 0 |
| African hydration complex | 0 | 0 | 0 | 3 |

TABLE 1B

| Activator | 1E | 1F | 1G | 1H |
|---|---|---|---|---|
| Composition | (wt. %) | (wt. %) | (wt. %) | (wt. %) |
| Deionized water | 66.7996 | 66.7996 | 66.7996 | 65.7996 |
| Carbomer | 2 | 2 | 2 | 3 |
| Guanidine carbonate | 28 | 28 | 28 | 28 |
| Glycerin | 2 | 2 | 2 | 2 |
| Red #33 | 0.0004 | 0.0004 | 0.0004 | 0.0004 |
| Aloe vera gel | 0.2 | 0.2 | 0.2 | 0.2 |
| Meadowquat ® HG-70 | 0 | 0 | 0 | 0 |
| DC silicone | 1 | 0 | 0 | 0 |
| Safflower seed oil | 0 | 1 | 0 | 0 |
| Sunflower oil | 0 | 0 | 1 | 0 |
| African hydration complex | 0 | 0 | 0 | 1 |

TABLE 1C

| Activator | 1I | 1J |
|---|---|---|
| Composition | (wt. %) | (wt. %) |
| Deionized water | 65.2996 | 65.0996 |
| Carbomer | 1.5 | 1.5 |
| Guanidine carbonate | 28 | 28.2 |
| Glycerin | 2 | 2 |
| Red #33 | 0.0004 | 0.0004 |
| Aloe barbadensis leaf juice | 0.2 | 0.2 |
| Meadowquat ® HG-70 | 3 | 3 |

### Example 2 (Comparative)

This example demonstrates methods for producing comparative activator compositions.

The lists of ingredients and the weight percentages for each of the Activators 2A-2C are summarized in Table 2. Activator 2D (which does not appear in Table 2) is the commercially available Dark & Lovely Relaxer Cream Activator, which contains water, petrolatum, paraffinum liquidum (mineral oil), guanidine carbonate, cetearyl alcohol, behentrimonium methosulfate, PEG-75 lanolin, polyquaternium 6, cocamidopropyl betaine, fragrance, butylphenyl methylpropional, propylene glycol, citronellol, geraniol, ci 15510 (orange 4), succinic acid, and denatonium benzoate.

Activator 2A is prepared as follows. Add a majority of the water (69.745350075 wt. % of the final wt. %) to a tank, and start mixing and recirculation (to keep the product in a state of constant motion to ensure homogeneous mixing). Add the sorbitol to the water. Slowly add the guanidine carbonate and denatonium benzoate with increased mixing. Rinse the tank with the remaining water (1.25 wt. % of the final wt. %) to flush all solids from the mixers and walls of the tank, and continue mixing for 120 minutes or until all solids are completely dissolved. If the percent of guanidine carbonate is determined to be high, mix the composition for additional time.

For Activator 2B, add the water to a tank, and start moderate mixing. Add the sorbitol. Heat the composition to 60-65° C. During heating, slowly add the guanidine carbonate and denatonium benzoate, continuing mixing for 60 minutes or until the guanidine carbonate and denatonium benzoate are completely dissolved. Cool the composition to 25-30° C. with moderate mixing.

For Activator 2C, add water (71.98589384 wt. % of the final wt. %) to a vessel. While mixing, heat the water to 60-65° C. During heating, slowly add the guanidine carbonate and denatonium benzoate. Once at 60-65° C., mix the composition for at least 60 minutes or until all of the guanidine carbonate and denatonium benzoate are dissolved. Cool the composition to 25-30° C. with moderate mixing. In a separate container, add water (1.00730616 wt. % of the final wt. %) and dissolve the green 5 and green 8 dyes with mixing. Add the dye composition to the main composition while mixing.

TABLE 2

| Activator | | | |
|---|---|---|---|
| Composition | 2A | 2B | 2C |
| (Comparative) | (wt. %) | (wt. %) | (wt. %) |
| Deionized water | 70.995350075 | 72.7491 | 72.9932 |
| Guanidine carbonate | 27.000531 | 27 | 27 |
| Sorbitol | 2.003183868 | 0.25 | 0 |
| (70% in water) | | | |
| Denatonium benzoate | 0.000935057 | 0.0009 | 0.0009 |
| Green 8 (CI 59040) | 0 | 0 | 0.0019 |
| Green 5 (CI 61570) | 0 | 0 | 0.004 |

### Example 3

This example illustrates methods for producing exemplary hydroxide compositions, which can be combined with the gel activator of the invention, as well as compositions that can be used before the relaxing treatment (as a pre-treatment) and after the relaxing treatment (as a post-treatment).

The lists of ingredients and the weight percentages thereof for Pre-Treatment Conditioners 3A and 3B are given in Table 3A, for Relaxer Bases 3A-3D in Table 3B, for Neutralizing Shampoos 3A and 3B in Table 3C, for a Post Neutralizing Conditioner in Table 3D, for a Masque Conditioner in Table 3E, and for a Leave-in Conditioner in Table 3F.

The Pre-Treatment Conditioner 3A is prepared as follows. Add a portion of the water (71.9% of the final wt. %) into a beaker. Add the disodium EDTA to the beaker, and mix the solution until the EDTA dissolves. Add the hydroxyethylcellulose, and mix the solution until the hydroxyethylcellulose is completely hydrated. Heat the solution to 75-80° C., and add the methylparaben, glycerin, cocamide MEA, polyquaternium-22 (40%), and PEG-12 dimethicone while heating. In a separate beaker, prepare the oil phase by adding the cetearyl alcohol, sorbitan oleate, ceteareth-20, oleth-5, oleth-20, coconut oil, and shea butter while heating to 75-80° C. After both beaker compositions reach 75-80° C., add the oil phase to the water phase, rinsing the oil phase beaker with additional hot water (5% of the final wt. %). Mix the composition for 15 minutes and then allow to cool. At 60° C., add the hydroxypropyltrimonium honey. At 45-48° C., individually add the phenoxyethanol, aloe vera, hydroxypropyl bisstearyldimonium chloride, and Salcare® SC-92 (polyquaternium-32 and mineral oil), mixing well after each addition. Add the fragrance, continuing mixing for a few more minutes.

The Pre-Treatment Conditioner 3B is prepared as follows. Into a stainless steel main compounding tank vessel, add cold deionized water (38.073% of the final wt. %) at 60-78° F./16-26° C. Sprinkle the hydroxyethylcellulose (HEC) into the main tank with vigorous mixing. Mix the mixture for a minimum of 20 minutes or until all the HEC is dispersed. Take one or more samples from the main processing vessel to verify that all of the HEC is dispersed. Make sure the powder is completely dispersed with no lumps present before the next steps are performed. Increase mixing as necessary to disperse any remaining particulates, and resample the mixture periodically until the batch is free from lumps. With vigorous mixing, add hot (at 175-185° F./80-85° C. where available) deionized water (35.742% of the final wt. %). As the batch level rises, temporarily reduce the turbine mixer speed as necessary to avoid splashing and aeration from the turbine blade(s). Resume vigorous mixing as soon as the batch level rises above the turbine blade(s). Mix the mixture for a minimum of 15 minutes (with a turbine mixer at a moderate counterclockwise direction and a side sweep mixer in a moderate forward direction). Heat the mixture to 175-185° F. (80-85° C.). Once above 145° F. (62° C.), add the following items: methyl paraben, glycerin, cocamide MEA, polyquaternium-22, behentrimonium chloride, and disodium EDTA/water. In a separate stainless steel phase vessel, add the following items to form an oil premix: cetearyl alcohol, sorbitan oleate/water, PEG-12 dimethicone, ceteareth-20, oleth-5, oleth-20, cocos nucifera (coconut) oil, and butyrospermum parkii (shea butter). Heat the premix to 175-185° F./80-85° C. When the temperatures of the main compounding tank and the oil premix are between 180-185° F./82-85° C. and all of the solids in both phases are completely melted, transfer the oil premix to the main compounding tank. Rinse and flush the premix vessel and transfer lines with deionized water (3.885% of the final wt. %), at 160-185° F./71-85° C. where available. Reduce the mixer speed to avoid splashing, and do not allow the mixture to settle out or cake on the tank walls. Mix the mixture for a minimum of 60 minutes (turbine mixer at vigorous counterclockwise direction, side sweep mixer at moderate forward direction), while holding it between 180-185° F./82-85° C. Cool the mixture to 94-97° F./34-36° C. with moderate mixing (turbine mixer at moderate counterclockwise direction, side sweep mixer at moderate forward direction). Add the hydroxypropyltrimonium honey once the temperature is below 140° F./60° C. When the batch temperature is below 113° F./45° C., add the following: phenoxyethanol, aloe barbadensis leaf juice, and fragrance (parfum) velocity 8111158. When the batch temperature is below 113° F./45° C., slowly add the polyquaternium-32/mineral oil/PPG-1 trideceth-6/water. Mix the mixture (turbine mixer at moderate to vigorous counterclockwise direction, side sweep mixer at moderate forward direction) for a minimum of 60 minutes or until all of the polyquaternium-32/mineral oil/PPG-1 trideceth-6/water is completely dissolved and a thick homogenous cream is formed. When the batch temperature is 95-99° F./35-37° C., sample the batch and discontinue mixing and cooling. Filter the mixture using an air diaphragm and positive displacement pump and 80 mesh filter, and fill into containers at 94-97° F./34-36° C.

For Relaxer Base 3A, add a majority of the water to a tank. Add the calcium hydroxide with vigorous mixing and recirculation. Continue mixing for 60 minutes or until all of the solids are dissolved. Add extra calcium hydroxide (up to 5% of total), if needed, based on activity measurements, to achieve the proper activity level. Use the remaining water (about 0.045 wt. %, up to the final wt. %) to rinse any calcium hydroxide adhering to the tank. Heat the composition to 82-85° C. with moderate agitation. Reduce mixing, and start de-aerating. Add the propylene glycol, mineral oil, petrolatum, and the pre-mixed wax blend of cetyl alcohol (35%), stearyl alcohol (50%), ceteth-20 (7.5%), and steareth-20 (7.5%) while mixing at 82-85° C. Mix the composition for at least 60 minutes and then cool. During cooling, increase the mixing speed to adjust for an increase in composition viscosity. When the temperature is below 45° C., add the fragrance (xenon CR II #109007-0195), the hydrolyzed vegetable protein PG-propyl silanetriol, and the aloe barbadensis leaf juice/algae extract mixture.

For Relaxer Base 3B, add the water to a tank. Add the calcium hydroxide and vigorously mix for at least 60 minutes. After complete dissolution of the calcium hydroxide, heat the composition to 80-85° C. When 80-85° C. is reached, add the following: the propylene glycol, the mineral oil, the petrolatum, and the pre-mixed wax blend as for Relaxer Base 3A. Mix the composition until all solids are melted and a smooth, homogenous consistency is attained. Continue mixing for at least 60 minutes. Start recirculation, and cool the composition. During cooling, increase the mixing speed to adjust for an increase in composition viscosity. When the temperature is below 45° C., add the fragrance (guava mango 086245-0793), aloe barbadensis leaf juice, and Panax ginseng root extract mixture.

For Relaxer Base 3C, add the petrolatum and mineral oil to a beaker and heat to about 82° C. During heating, add a pre-mixed wax blend as for Relaxer Base 3A. In a separate container, add water (29 wt. % of the final wt. %) and the propylene glycol, and heat to 85° C. After both the water and oil phases reach about 82° C., transfer the water phase into the oil phase beaker. Continue mixing for 15 minutes, and then start cooling. In another separate container, add water (14 wt. % of the final wt. %) and the calcium hydroxide, and mix until the calcium hydroxide is completely dissolved. Add the calcium hydroxide mixture to the oil mixture when the oil mixture reaches 35° C. Place the beaker in an ice bath for 15 minutes.

For Relaxer Base 3D, add the petrolatum and mineral oil to a beaker, and heat to about 82° C. During heating, add the cetyl alcohol/stearyl alcohol/ceteth-20/steareth-20. In a separate container, add water (29 wt. % of the final wt. %) and the propylene glycol, and heat to 85° C. After both the water and oil phases reach about 82° C., transfer the water phase into the oil phase beaker. Continue mixing for 15 minutes, and then start cooling. Once the temperature is below 50° C., add the fragrance (velocity). In another separate container, add water (13.8 wt. % of the final wt. %) and the calcium hydroxide, and mix until the calcium hydroxide is completely dissolved. Add the calcium hydroxide mixture to the oil/water mixture when the oil/water mixture reaches 35° C. Place the beaker in an ice bath for 15 minutes.

Another method for preparing Relaxer Base 3D is as follows. Place the petrolatum, if stored in a drum, in a hot room prior to its use. Check the activity of the calcium hydroxide prior to addition, and accordingly adjust the amount if needed. For upfront adjustment only, if activity is low, a maximum of 5% of the total calcium hydroxide can be added to the formula. Into a main stainless steel compounding tank, add cold (at 40-60° F./4-15° C. where available) deionized water (42.8% of the final wt. %, minus 100 pounds for rinsing after addition of calcium hydroxide). Add the calcium hydroxide with vigorous mixing, vortexing where available. Start a recirculation loop from bottom to top. Continue mixing (turbine mixer at vigorous counterclockwise direction, side sweep mixer at moderate forward direction), for a minimum of 60 minutes. Take a sample to verify that all of the solids are dissolved. Resample periodically to determine if there are any undissolved or undispersed materials. Once fully dissolved, heat the mixture to 180-185° F./82-85° C. with moderate agitation. Reduce mixing to begin de-aerating the batch. When the batch temperature is between 176-185° F./80-85° C., add the following to the main processing tank with mixing (turbine (inside) mixer at moderate counterclockwise direction, side sweep mixer at moderate forward direction): propylene glycol, mineral oil, petrolatum, and cetyl alcohol/stearyl alcohol/ceteth-20/steareth-20. Mix the mixture for a minimum of 60 minutes (turbine mixer at vigorous counterclockwise direction, side sweep mixer at moderate forward mixing (turbine (inside) mixer at moderate counterclockwise direction, side sweep mixer at moderate forward direction). Reduce the mixer speed to avoid splashing, and do not allow the mixture to settle out or cake on the tank walls. However, as the batch viscosity increases when below 122° F./50° C., increase the mixing to adjust for the viscosity rise. When the batch temperature is below 113° F./45° C., add the Fragrance (Parfum) Velocity 8111158 to the main compounding tank. Mix the mixture for a minimum of 120 minutes (turbine mixer at moderate counterclockwise direction, side sweep mixer at moderate forward direction). When the batch is completely uniform and the temperature is between 73-77° F./23-25° C., remove a sample from the recirculation line, and reduce the sweep mixer (side sweep mixer at slow forward direction), and turn off the turbine mixer and recirculation. Cool as needed to maintain the temperature at 73-77° F./23-25° C. Take additional samples and test. With the batch temperature at 73-77° F./23-25° C., turn on the recirculation loop, and take in-line PD, in-line HSD, and milled samples. After the samples are taken, stop the recirculation. Mill the batch into a holding tank for filling. Turn on the sweep mixer at minimum speed (side sweep mixer at slow forward direction), and mill the batch into portables. Filter the mixture using an air diaphragm and positive displacement pump and 80 mesh filter, and fill into containers at 73-77° F./23-25° C.

Relaxer Base 3E is the commercially available Dark & Lovely Relaxer Base.

The following procedure is used to prepare the Neutralizing Shampoo 3A. Store the sodium C14-C17 alkyl sec sulfonate in a hot room, and mix thoroughly prior to use. Add a portion of the water (26.348999508 wt. % of the final wt. %) to a tank. Start vigorous mixing, and add the polyquaternium-10, mixing for at least 60 minutes until the polyquaternium-10 is completely dispersed. Heat the composition to 60-63° C. with moderate agitation, and add the following during heating: water (21.959813465% of the final wt. %), the cocamidopropyl betaine, the cocotrimonium chloride, and the PEG-120 methyl glucose dioleate. Once 60-63° C. is attained, add the PPG-5-ceteth-10 phosphate and sodium C14-17 alkyl sec sulfonate with moderate agitation. Continue mixing for at least 30 minutes. Cool the composition to 35-38° C. with moderate agitation. During cooling, add cold water (14.886219704% of the final wt. %), the sodium laureth sulfate, disodium cocoamphodipropionate, and PEG-12 dimethicone. In a separate container, add the PEG-30 glyceryl cocoate and fragrance (fresh peach 26358), and mix until the mixture is clear and transparent. Add the separate mixture to the tank when the separate mixture is clear and transparent and the tank mixture is below 45° C. Add the aloe barbadensis leaf juice/algae extract mixture, hydrolyzed vegetable protein PG-propyl silanetriol, and the DMDM hydantoin/iodopropynyl butylcarbamate mixture. In another separate container, mix water (1.055760263% of the final wt. %) with the citric acid until the citric acid is completely dissolved. Add the citric acid mixture to the tank after the tank mixture is below about 37° C. In yet another separate container, mix water (0.04500706% of the final wt. %) with the phenolsulfonphthalein until complete dissolution. Add this mixture to the tank with moderate agitation after the tank is below 45° C. Mix the composition for at least an additional 30 minutes. Adjust the pH of the composition by adding citric acid (to a maximum of 5% of the total wt. % citric acid) if the pH is too high. Adjust the viscosity of the composition by adding cocamidopropyl betaine (to a maximum of 5% of the total wt. % cocamidopropyl betaine) if the viscosity is too low.

For the Neutralizing Shampoo 3B, add water (26.096% of the final wt. %) to a main tank. Disperse the polyquaternium-10 in the water with agitation, and mix for 20 minutes, after which, heat the mixture to 60° C. Add additional water (22% of the final wt. %) and the cocamidopropyl betaine, cocotrimonium chloride, PEG-120 methyl glucose dioleate, PPG-5-ceteth-10 phosphate, and sodium C14-17 alkyl sec sulfonate, in the order listed. Mix the contents of the main tank for 15 minutes at 60° C. and then cool to 38° C. Add more water (15% of the final wt. %), the sodium laureth sulfate, disodium cocoamphodipropionate, and PEG-12 dimethicone, in the order listed. Prepare a pre-mix by mixing the PEG-30 glyceryl cocoate and the fragrance (velocity 8111158). Add the fragrance pre-mix to the main tank when the main tank reaches 38° C. or below. Add the DMDM hydantoin to the main tank, also when the main tank is at or below 38° C. Prepare another pre-mix by mixing water (1% of the final wt. %) and the citric acid. Add the citric acid pre-mix to batch at or below 38° C. Prepare a final pre-mix by mixing water (0.05% of the final wt. %) and the phenol red. Add the final premix to the main tank at or below 38° C.

An alternative method for preparing Neutralizing Shampoo 3B follows. Store the C14-C17 Alkyl Sec Sulfonate in a hot room, and thoroughly mix prior to use. Into a stainless steel main compounding tank, add the deionized water (26.236293% of the final wt. %), cold at 40-60° F./4-16° C. where available. Stir the water with vigorous stirring (vortexing where available). Add the polyquaternium-10, and use a high shear disperser, where available. Mix the mixture for a minimum of 60 minutes (turbine mixer at vigorous, side sweep mixer at moderate forward direction). Take a sample of the main compounding tank to verify that all of the polyquaternium-10 is dispersed with no lumps or particulates. Visually verify that the main compounding tank is free from undispersed polyquaternium-10. Increase the mixing speed as necessary to disperse any remaining particulates, and resample the batch periodically until the batch is free from particulates. Heat the batch to 140-145° F./60-63° C. with moderate agitation. Add the following items with moderate agitation: water (hot, where available, 21.908650 of final wt. %), cocamidopropyl betaine/water, cocotrimonium chloride/water, and PEG-120 methyl glucose dioleate. When the batch temperature is between 140-145° F./60-63° C., add the PPG-5-ceteth-10 phosphate and sodium C14-17 alkyl sec sulfonate/water to the main compounding tank with moderate agitation. Mix the mixture for a minimum of 30 minutes (turbine mixer at moderate, side sweep mixer at moderate forward direction), holding between 140-145° F./60-63° C. Cool the batch to 94-99° F./35-38° C. with moderate agitation. Once the cooling is initiated, add the following items to the main compounding tank: deionized water (cold where available, 14.851537% of final wt. %), sodium laureth sulfate 1.0 mole/water, disodium cocoamphodipropionate/water, and PEG-12 dimethicone. Prepare a premix by adding the PEG-30 glyceryl cocoate and fragrance (parfum) velocity 8111158 into a separate stainless steel kettle or phase vessel. Blend the premix until all is solubilized and the premix is completely clear and transparent. Once the premix is completely clear and transparent and the batch temperature is below 113° F./45° C., the add the premix to the main compounding tank. With the batch temperature below 113° F./45° C., add the DMDM hydantoin/water to the main compounding tank with moderate agitation. Prepare a citric acid premix. Into a stainless steel kettle or phase vessel, add the citric acid and water (1.0533% of the final wt. %), and blend until the citric acid is completely dissolved. Once the batch temperature is below 99° F., add the citric acid premix to the main compounding tank. Prepare a final premix by adding the phenolsulfonphthalein and water (0.096219% of the final wt. %) into a stainless steel kettle or premix vessel. Visually inspect the premix to verify that the dye is completely dissolved before adding it to the main compounding tank. Once the dye premix is verified and the batch temperature is below 113° F./45° C., add the dye premix to the batch with moderate agitation. Mix the batch for a minimum of 30 minutes (turbine mixer at moderate, side sweep mixer at moderate forward direction). When the batch temperature is 94-99° F./34-37° C., sample the batch, and discontinue mixing and cooling. Filter the mixture using an air diaphragm and positive displacement pump and 80 mesh filter, and fill into containers at 94-99° F./34-37° C.

The following adjustments are made, if necessary. If the pH is high, citric acid, at a maximum of 5% of the total batch requirement, can be added. If the viscosity is low, cocamidopropyl betaine, at a maximum of 5% of the total batch requirement, can be added.

The Post Neutralizing Conditioner is prepared as follows. Add the water into a beaker. Add the polyquaternium-32/mineral oil/PPG-1 trideceth-6 mixture, and mix until a homogenous cream is formed. Add the mineral oil, and mix until all of the mineral oil is mixed into the cream. Add the following, mixing well after each addition: dimethicone, dimethicone PEG-7 isostearate, quaternium-80/propylene glycol mixture, glycerin, shea butter, and coconut oil. Continue mixing until creamy and homogenous. Add the DMDM hydantoin, panthenol, honey, wheat proteins and fragrance (velocity), and mix for a few more minutes.

For the Masque Conditioner 3A, heat the water to 82-85° C. Add the citric acid and disodium EDTA (99%)/water (1%). Add the quaternium-18 (96%)/water (4%) with vigorous mixing for 15 minutes. Individually add the stearamidopropyl dimethylamine, cetyl alcohol, and stearyl alcohol in order, mixing well after each addition. Add the cocoa butter, honey, and coconut oil, and continue mixing for 30 minutes. Once homogenous, reduce the mixing speed to de-aerate, and continue mixing for 10 minutes. Cool the composition to 60° C., very slowly sprinkle in the aspartic acid, and continue mixing until the aspartic acid is completely dissolved. Mix the composition for an additional 45 minutes at 60° C. Once it is determined there are no particulates in the composition, decrease the mixing speed, and add the Isostearamidopropyl ethyldimonium ethosulfate (75%)/PEG-9 (15%) mixture. Cool the composition to 45° C., and add the silk amino acids, DMDM hydantoin (55%)/water (45%), Chamomilla Recutita (Matricaria) flower extract (10%)/propylene glycol (27%)/water (61.9%) mixture, panthenol, polyglyceryl-3 distearate mixture (Water (48%), polyglyceryl-3 distearate (25%), polysorbate 60 (10%), myristic acid (2%), palmitic acid (2%), stearic acid (2%), guar hydroxypropyltrimonium chloride (2%), Triticum Vulgare (Wheat) Flour Lipids (<1%), Ceramide 3 (<1%), Persea Gratissima (Avocado) Oil (<1%), Tetrahexyldecyl Ascorbate (<1%), Glycine Soja (Soybean) Sterols (<1%), Alpha-Glucan Oligosaccharide (<1%), Ethylhexyl Methoxycinnamate (<1%), Butyl ethoxydibenzoylmethane (<1%), Tocopheryl Acetate (<1%)), fragrance (velocity), and the cyclopentasiloxane (70%)/cyclohexasiloxane (15%)/dimethicone (15%) mixture. Mix the composition for 60 minutes. The fragrance of the Masque Conditioner can be reduced to 0.3 wt. % with an increase in water by 0.2 wt. %.

The Masque Conditioner 3B is prepared as follows. Into a main processing tank, add the deionized water at 180-185° F./82-85° C., where available. Heat the batch as necessary to 180-185° F. (82-85° C.) to maintain temperature. Stir the water (turbine mixer at moderate counterclockwise direction, side sweep mixer (where available) in clockwise direction). During heating, add the citric acid and disodium EDTA/water. While maintaining the batch temperature between 180-185° F./82-85° C., add the quaternium-18/water with vigorous mixing. Mix the batch for a minimum of 15 minutes or until the batch is homogeneous and free of particles (turbine mixer at vigorous counterclockwise direction, side sweep mixer at moderate clockwise direction). Visually verify that the batch is free of particles before continuing. Maintaining the batch temperature between 180-185° F./82-85° C., add the following ingredients in order to the main compounding tank with moderate agitation, mixing well between additions: stearamidopropyl dimethylamine, cetyl alcohol, stearyl alcohol, theobroma cacao (cocoa) seed butter, and cocos nucifera (coconut) oil. Mix the batch for a minimum of 30 minutes (turbine mixer at vigorous counterclockwise direction, side sweep mixer at clockwise direction), while holding the temperature between 180-185° F./82-85° C. Take a sample, and visually inspect the main compounding tank to verify that all of the solids are melted and that the batch is smooth and homogeneous. Continue vigorous mixing until all of the waxes and particulates are dissolved and the batch is homogeneous. Reduce the turbine and side sweep mixer speeds to moderate/slow for a maximum of 10 minutes to aid in de-aeration of the batch. Avoid immediate cooling to prevent entrapping air. Mix the batch for a maximum of 10 minutes or just until the batch appears uniform and free of excess air (turbine mixer at moderate counterclockwise direction, side sweep mixer at slow clockwise direction). After the de-aeration step, cool the batch to 135-140° F./57-60° C. with moderate mixing. As the batch begins to cool, increase the mixer speeds to insure good turnover of the batch. When the batch temperature reaches between 135-140° F./57-60° C., turn off the cooling, and sprinkle in the aspartic acid into the main tank, maintaining vigorous agitation. Addition of the aspartic acid takes at least 10-15 minutes. Mix the batch for a minimum of 45 minutes or until all solids are dissolved and the batch is smooth and homogeneous (turbine mixer at vigorous counterclockwise direction, side sweep mixer at clockwise direction). To avoid particles in the batch, dissolve the aspartic acid completely before the batch temperature cools below 135° F. (57° C.). Sample the batch and visually inspect to verify the batch is free from any undissolved materials or particulates. If particulates are present, maintain the batch above 135° F. and mix until the batch is free of particulates. When no particulates are present, add the isostearamidopropyl ethyldimonium ethosulfate/PEG-9, and cool the batch to 99-102° F./37-39° C. with moderate to vigorous mixing. Adjust the mixing speeds to avoid aeration since the viscosity decreases after this addition. When the batch temperature is below 113° F./45° C., add the following to the main compounding tank: cyclopentasiloxane/cyclohexasiloxane/dimethicone, fragrance (parfum) velocity 8111158, DMDM hydantoin/water, silk amino acids, honey, chamomilla recutita (matricaria) flower extract, propylene glycol/water, panthenol, and water/polyglyceryl-3 distearate/polysorbate 60/myristic acid/palmitic acid/stearic acid mixture. Mix the batch for a minimum of 60 minutes (turbine mixer at vigorous clockwise direction—for the first 10 minutes or until isostearamidopropyl ethyldimonium ethosulfate/PEG-9 is incorporated, then vigorous counterclockwise direction for the remainder of the mixing time, side sweep mixer at clockwise direction). When the batch temperature is 99-104° F./37-40° C., take a sample, and discontinue mixing and cooling.

The following adjustments are made, if necessary. If the viscosity is high, isostearamidopropyl ethyldimonium ethosulfate/PEG-9 can be added. If the viscosity is low, take a sample, and pump it through an 80 mesh and paper filter. If the viscosity is still low, a pump sample can be pulled through a Silverson mill to determine additional modification. If the pH is high, citric acid can be added as necessary.

Filter the mixture using an air diaphragm and positive displacement pump and 80 mesh filter, and fill into containers at 95-104° F./35-40° C. Cool the batch, as necessary, but do not reheat.

The Leave-In Conditioner is prepared as follows. Into a main stainless steel compounding tank, add deioinzed water (92.576118% of the final wt. %), warm (95-104° F./35-40° C.), where available. Do not heat the water to maintain temperature. With moderate mixing, add the following ingredients to the main compounding tank, mixing well after each addition: amodimethicone/trideceth-12/cetrimonium chloride/water, glycerin, PEG-12 dimethicone, propylene glycol, PVP, DMDM hydantoin/water, cetrimonium chloride/water, benzophenone-4, disodium EDTA/water, triethanolamine, cocos nucifera (coconut) oil, water/polyglyceryl-3 distearate/polysorbate 60/myristic acid/palmitic acid/stearic acid mixture, and water/propylene glycol/honey extract. Mix the batch for a minimum of 20 minutes (turbine mixer at moderate, side sweep mixer at moderate forward direction). Into a separate stainless steel phase vessel, prepare a premix by adding the fragrance (parfum) velocity 8111158 and polysorbate 20. With the use of an electric or air operated mixer, blend the premix until all of the fragrance is solubilized and the premix is homogeneous. Once homogenous, add the premix to the main compounding tank with moderate agitation. Rinse the premix vessel with water (0.746582% of the final wt. %), cold (65-70° F./18-21° C.), where available. Add the rinse water to the main processing tank with moderate agitation. Mix the batch for a minimum of 30 minutes (turbine mixer at moderate, side sweep mixer at moderate forward direction). Take samples, and discontinue mixing. If the pH is low, add triethanolamine at 0.20% solution increments. Filter the mixture using an air diaphragm and positive displacement pump and 80 mesh filter, and fill into containers at 75-95° F./24-35° C. Cool the batch as necessary to facilitate filling.

TABLE 3A

| | Pre-Treatment | Pre-Treatment |
|---|---|---|
| | Conditioner 3A | Conditioner 3B |
| Composition | (wt. %) | (wt. %) |
| Deionized Water | 76.9 | 77.7 |
| Cetearyl Alcohol | 2.5 | 2.5 |
| Polyquaternium-22 | 2.25 | 2.25 |
| PEG-12 Dimethicone | 2 | 2 |
| Sorbitan Oleate | 2 | 2 |
| Salcare ® SC-92 | 2 | 0 |
| Polyquaternium-32, mineral oil, PPG-1 | 0 | 2 |
| trideceth-6, water | | |
| Glycerin | 1.5 | 1.5 |
| Cocamide MEA | 1.5 | 1.5 |
| Hydroxypropyltrimonium Honey | 1.5 | 1.5 |
| Hydroxypropyl Bisstearyldimonium | 1.5 | 0 |
| Chloride | | |
| Ceteareth-20 | 1 | 1 |
| Oleth-5 | 1 | 1 |
| Oleth-20 | 1 | 1 |
| Coconut Oil (cocos nucifera oil) | 1 | 1 |
| Phenoxyethanol | 0.8 | 0.8 |
| Fragrance | 0.6 | 0.3 |
| Hydroxyethylcellulose | 0.5 | 0.5 |
| Methylparaben | 0.2 | 0.2 |
| Shea butter (butyrospermum parkii) | 0.1 | 0.1 |
| Aloe Vera | 0.1 | 0 |
| Disodium EDTA | 0.05 | 0.05 |
| Aloe barbadensis leaf juice | 0 | 0.1 |
| Behentrimonium chloride | 0 | 1 |

TABLE 3B

| Relaxer Base | 3A | 3B | 3C | 3D |
|---|---|---|---|---|
| Composition | (wt. %) | (wt. %) | (wt. %) | (wt. %) |
| Deionized water | 42.7998 | 42.2999 | 43 | 42.8 |
| Petrolatum | 22 | 22 | 22 | 22 |
| Mineral oil | 14 | 14 | 14 | 14 |
| Cetyl alcohol, stearyl alcohol, | 10 | 10 | 10 | 10 |
| ceteth-20, steareth-20 | | | | |
| Calcium hydroxide | 6 | 6 | 6 | 6 |
| Propylene glycol | 5 | 5 | 5 | 5 |
| Water, aloe barbadensis leaf juice, | 0.0001 | 0.5 | 0 | 0 |
| algae extract | | (leaf | | |
| | | juice | | |
| | | only) | | |
| Hydrolyzed vegetable protein PG- | 0.0001 | 0 | 0 | 0 |
| propyl silanetriol (15%), | | | | |
| water (85%) | | | | |
| Fragrance | 0.2 | 0.2 | 0 | 0.2 |
| Panax ginseng root extract (6.6%), | 0 | 0.0001 | 0 | 0 |
| Chamomilla Recutita (Matricaria) | | | | |
| flower extract (6.6%), aloe | | | | |
| barbadensis leaf juice (0.33%), | | | | |
| mineral oil (86.47%) | | | | |

TABLE 3C

| | Neutralizing | Neutralizing |
|---|---|---|
| | Shampoo 3A | Shampoo 3B |
| Composition | (wt. %) | (wt. %) |
| Deionized water | 64.2958 | 64.146 |
| Sodium laureth sulfate (30% in water) | 18 | 18 |
| Cocamidopropyl betaine (37% in water) | 10.9 | 10.9 |
| Disodium cocoamphodipropionate (40% in | 1.5 | 1.5 |
| water) | | |
| Sodium C14-17 alkyl sec sulfonate (60% in | 1 | 1 |
| water) | | |
| PEG-30 glyceryl cocoate | 1 | 1 |
| PPG-5-ceteth-10 phosphate | 0.75 | 0.75 |
| Fragrance | 0.7 | 0.8 |
| Cocotrimonium chloride (34% in water) | 0.55 | 0.55 |
| Polyquaternium-10 | 0.4 | 0.4 |
| DMDM hydantoin (70%), iodopropynyl | 0.3 | 0.3 |
| butylcarbamate (2.6%), water (27.4%) | | |
| PEG-12 dimethicone | 0.25 | 0.25 |
| PEG-120 methyl glucose dioleate | 0.2 | 0.2 |
| Citric acid, USP anhydrous granular | 0.15 | 0.2 |
| Phenolsulfonphthalein | 0.004 | 0.004 |
| Water, aloe barbadensis leaf juice, algae | 0.0001 | 0 |
| extract | | |
| Hydrolyzed vegetable protein PG-propyl | 0.0001 | 0 |
| silanetriol (15% in water) | | |

TABLE 3D

| | Post Neutralizing |
|---|---|
| | Conditioner |
| Composition | (wt. %) |
| Deionized water | 87.9 |
| Polyquaternium-32 (54%), mineral oil (30%), PPG-1 | 4.4 |
| trideceth-6 (6%) water (10%) | |
| Mineral oil | 3 |
| Glycerin | 2 |
| Quaternium-80 (51%), propylene glycol (49%) | 0.5 |
| Dimethicone PEG-7 isostearate | 0.5 |
| Dimethicone | 0.5 |
| DMDM hydantoin | 0.3 |
| Fragrance | 0.3 |
| Panthenol | 0.2 |
| Shea butter | 0.1 |
| Coconut oil | 0.1 |
| Honey | 0.1 |
| Wheat proteins | 0.1 |

TABLE 3E

| | Masque | Masque |
|---|---|---|
| | Conditioner | Conditioner |
| Composition | 3A (wt. %) | 3B (wt. %) |
| Deionized water | 82.5347 | 82.7347 |
| Stearyl alcohol | 5.286 | 5.286 |
| Cyclopentasiloxane, cyclohexasiloxane, | 5 | 5 |
| dimethicone | | |
| Cetyl alcohol | 2.714 | 2.714 |
| Stearamidopropyl dimethylamine | 2 | 2 |
| Aspartic acid | 0.6 | 0.6 |
| Quaternium-18, water | 0.5 | 0.5 |
| Fragrance | 0.5 | 0.3 |
| DMDM hydantoin, water | 0.2 | 0.2 |
| Isostearamidopropyl ethyldimonium | 0.145 | 0.145 |
| ethosulfate, PEG-9 | | |
| Silk amino acids | 0.1 | 0.1 |
| Citric acid, USP anhydrous granular | 0.1 | 0.1 |
| Cocoa butter (Theobroma cacao seed butter) | 0.1 | 0.1 |
| Honey | 0.1 | 0.1 |
| Coconut oil (Cocos nucifera oil) | 0.1 | 0.1 |
| Disodium EDTA, water | 0.02 | 0.02 |
| Chamomilla Recutita (Matricaria) flower | 0.0001 | 0.0001 |
| extract, propylene glycol, water | | |
| Panthenol | 0.0001 | 0.0001 |
| Water, polyglyceryl-3 distearate, polysorbate | 0.0001 | 0.0001 |
| 60, myristic acid, palmitic acid, stearic acid, | | |
| guar hydroxypropyltrimonium chloride, | | |
| Triticum Vulgare (Wheat) Flour Lipids, | | |
| Ceramide 3, Persea Gratissima (Avocado) | | |
| Oil, Tetrahexyldecyl Ascorbate, Glycine | | |
| Soja (Soybean) Sterols, Alpha-Glucan | | |
| Oligosaccharide, Ethylhexyl | | |
| Methoxycinnamate, Butyl | | |
| ethoxydibenzoylmethane, Tocopheryl | | |
| Acetate | | |

TABLE 3F

| | Leave-in |
|---|---|
| | Conditioner |
| Composition | (wt. %) |
| Water | 93.3227 |
| Amodimethicone, trideceth-12, cetrimonium | 2 |
| chloride, water | |
| Cetrimonium chloride, water | 1.2 |
| Glycerin | 1 |
| Peg-12 dimethicone | 0.5 |
| Propylene glycol | 0.5 |
| PVP | 0.45 |
| DMDM hydantoin, water | 0.3 |
| Polysorbate 20 | 0.3 |
| Fragrance (parfum) velocity 8111158 | 0.200 |
| Benzophenone-4 | 0.1 |
| Disodium EDTA, water | 0.1 |
| Triethanolamine | 0.0270 |
| Cocos nucifera (coconut) oil | 0.0001 |
| Water, propylene glycol, honey extract | 0.0001 |
| Water, polyglyceryl-3 distearate, polysorbate 60, | 0.0001 |
| myristic acid, palmitic acid, stearic acid, guar | |
| hydroxypropyltrimonium chloride, Triticum Vulgare | |
| (Wheat) Flour Lipids, Ceramide 3, Persea Gratissima | |
| (Avocado) Oil, Tetrahexyldecyl Ascorbate, Glycine Soja | |
| (Soybean) Sterols, Alpha-Glucan Oligosaccharide, | |
| Ethylhexyl Methoxycinnamate, Butyl | |
| ethoxydibenzoylmethane, Tocopheryl Acetate | |

### Example 4

This example demonstrates the reduction in hair breakage achieved by using an exemplary relaxer gel activator of the invention relative to comparative activators.

African American Kinky Virgin Hair tresses (purchased from Demeo Brothers, New York, N.Y.) were prepared. Each assembly was dark brown to black in color, pre-stitched with a paraffin tab at the root, five inches wide, and 3-5 inches long before relaxing (which corresponded to 6-8 inches long after relaxing). One inch wide swatches were prepared by cutting the tresses, and two swatches were attached together using a glue gun and duct tape wrapped around once. Each swatch weighed approximately 7-9 grams.

Sixteen swatches were used per comparison with eight swatches used as test swatches and eight swatches used as control swatches, depending upon the activator (see Table 4). For the Comparisons 1-4, Activators 1A and 1B and Activators 2A and 2B were each individually mixed with the Relaxer Base 3A until the mixture was homogeneous. For the Comparisons 3 and 4, Activator 2D was mixed with Relaxer Base 3E according to the manufacturer's instructions regarding mixing the activator with base. For all comparisons, 213 g of relaxer base was mixed with 60 g of activator to produce the relaxer mixture.

Approximately 25 grams of each relaxer mixture was applied to entirely cover separate swatches. Within 15 minutes of application, the relaxer was smoothed (using the back end of a standard comb) through the end of the swatch. Two smoothing sessions were performed on each swatch. This smoothing technique was used to straighten the hair out as much as possible, removing loose hairs from the swatch as smoothing was performed until the 15 minutes elapsed. The swatch was rinsed thoroughly for at least 5 minutes with luke-warm water until there was no visible trace of relaxer on the swatch. All tresses were then neutralized following the above treatments using the Neutralizing Shampoo 3A, completing the treatment cycle.

Tresses were air dried overnight at ambient temperature and flogged one time (1×) the next day using a mechanical comb. The flogger settings were a control speed of seven with the counter set to 100.

Broken hair fibers were collected during flogging and counted immediately following the flogging session. The total number of broken hairs resulting from flogging was totaled together for each set of eight test and control swatches. The average percent reduction in fiber breakage from mechanical damage of the test activator compared to the control activator was calculated: (((# of fibers broken for control tress)−(# of fibers broken for treatment tress))/(# of fibers broken for control tress)×100)/(8 tresses). To determine the strengthening effect, the magnitude of breakage was also evaluated by calculating the ratio of the number of broken fibers for the control set to the number of broken fibers during the test set: (total control)/(total treatment). An n-fold reduction in fiber breakage was considered to imply an n-fold increase in fiber strength. P-values were calculated in Microsoft Excel's Analyse It using the Signed Rank Test (a non-parametric method of analyzing the difference between two samples) to determine whether significant differences in total fiber breakage existed between the control and test sets.

The results of the experiments are given in Table 4.

TABLE 4

| | | | Average | | |
|---|---|---|---|---|---|
| | | | % | | |
| | | | Reduction | n-Fold | |
| | | | in | Im- | Significance |
| Com- | Treatment | Control | Fiber | prove- | of Data |
| parison | Activator | Activator | Breakage | ment | (P-value) |
| 1 | 1A | 2A | 13.24 | 1.3 | No (P = 1.00) |
| 2 | 1B | 2B | 60.67 | 2.8 | Yes (P = 0.0078) |
| 3 | 1A | 2D | −35.97 | 0.8 | No (P = 0.2891) |
| 4 | 1B | 2D | 39.02 | 1.7 | Yes (P = 0.0078) |

The results of Table 4 show that the use of carbomer and more than a trace amount of meadowquat (3% in Activator 1B) provides superior reduction in fiber breakage.

### Example 5

This example demonstrates the overall performance achieved by exemplary relaxer gel activators of the present invention.

The half-head evaluation method was used, where one activator was applied to one half of a subject's head and another activator was applied to the other half of the same subject's head. All subjects had African-American hair type. Activators 1C and 2C were compared per head after mixing with Relaxer Base 3B (Table 5A). The following activators were compared per head after mixing with Relaxer Base 3C: Activators 1B and 1C (Table 5B), 1C and 1D (Table 5C), 1C and 1E (Table 5D), 1C and 1H (Table 5E), 1C and 1F (Table 5F), and 1C and 1G (Table 5G).

The following procedures were used. The subject completed a relaxer checklist to help determine any cause of burning or irritation during the tests. The stylists checked the subjects' scalps for redness, abrasions, and/or pimples. The stylists also checked the subjects' hair for breakage, texture, porosity, and overall condition to determine relaxer time. If the scalp was clear and the hair met all testing criteria, the test continued.

To mix the activator and relaxer base, 60 g of the activator was added to 213 g of the base and mixed with a wooden spatula with a circular motion. For a half-head application, about 7 ounces of the resulting mixture was applied, but the final amount varied due to the amount of hair. The hair was at least three inches long, and the new growth was covered with the mixture.

The hair was divided into four equal sections, using the cross method, parting the hair in the middle and from ear to ear. A timer was set to 18 minutes for total processing time (Activators 1B-1H) or the maximum manufacturer's suggested time (Activator 2C). Application for each section took about 2.5 to 5 minutes. The mixture was applied starting on the back right section in the nape area 0.5 inches from the nape, moving up to the crown of the head. The mixture was applied around the perimeter of the sections using a brush. The sections then were subdivided into ¼ inch to ⅛ inch partings with the tail of the application brush. For each parting, the mixture was applied ⅛ of an inch from the scalp area. This procedure was repeated for the front right section, leaving the hairline for last, and then was repeated for the back left section and front left section, again leaving the hairline for last.

After the relaxer was applied to all sections, the hair was smoothed by section in the order in which the mixture was applied. The brush or back of a finger was used by making one downward stroke in each subsection. Holding the section of hair in an upward taunt position for control, the hair was sectioned and smoothed downward in one stroke.

After the smoothing, the relaxation of the hair was checked to determine whether both sides equally reached the appropriate straightness. If the hair had not reached the desired straightness and additional time was available (up to 18 minutes (Activators 1B-1H) or the maximum suggested processing time (Activator 2C)), the additional time was used. Once the hair was straight, and/or the time limit was reached, the stylists proceeded to the next step. However, if the subject had irritation that was more than a spot that could be sprayed with oil sheen to sooth it, the hair was rinsed at that time to remove the relaxer, beginning with the irritated area and then following the application of the mixture.

The hair sections then were rinsed in the order of relaxer application using water as warm as possible, up to the subject's comfort level. Rinsing was continued until all of the relaxer had been removed. While rinsing, the hair was lifted to insure that the water hit on the scalp and removed the relaxer. After rinsing, the hair and scalp were examined to make sure the relaxer had been removed. The subject's neck was cleaned using a towel, and the towel was changed before continuing.

The hair was shampooed with the Neutralizing Shampoo 3A (Table 3C), distributing the shampoo throughout the head with gentle manipulation. Three shampoo cycles were performed, after which the hair was conditioned with the Masque Conditioner 3A (Table 3E). The hair then was dried simultaneously using two similar brushes using the same pattern on both sides.

Observations by the stylist were made on the following criteria. Application: The product did not drip or run during application and had a good consistency so that the stylist could maintain control when placing and distributing the product on the hair in the desired manner. Breakdown: The hair maintained a straight pattern upon manipulation. Rinseability: Ease of rinsing the mixture away. Irritation: The scalp and hairline remained in a clear condition. The product had not caused any irritation, burning, itching, redness or any adverse effect on the scalp and/or hairline. Straightness: The hair's natural curl had the desired straightness, ideally the hair was not so straight to the point of breakage, or flat where there was no body to the hair. The following factors were considered when evaluating straightness: processing time, the formula strength (e.g., super, normal, mild), the condition of the hair (e.g., dry, normal, oily), and the texture of the hair (fine, medium, coarse). Feel After Towel Drying: The hair was felt from the scalp to the ends of the hair to determine if the hair was pliable and smooth to the touch. Sheen After Drying: This was evaluated by the stylists standing stationary and observing the reflection of light from the hair while turning the subject's chair from side to side. Manageability: The hair was flexible and easy to style. Overall preference: This was based on how beneficial the product was to the hair. Finally, comments were made when necessary to complete the evaluation or clarify an evaluation.

Results were determined by the observations of the stylist according to the following scale: 1 for poor, 2 for fair, 3 for good, 4 for very good, and 5 for excellent. The stylist asked the subject regarding irritation and used a different scale: 1 for no irritation to 5 for much irritation. Tables 5A-5G present the average data for the given number of subjects, including the overall preference given by the subjects.

TABLE 5A
No. of subjects = 3.

| | | Activator 2C |
|---|---|---|
| | Activator 1C | (Comparative) |
| Application | 4.3 | 5 |
| Breakdown | 4.3 | 4.3 |
| Rinseability | 3.3 | 5 |
| Odor | 4.7 | 4.7 |
| Irritation | 1 | 1 |
| Straightness after towel drying | 3 | 4.7 |
| Feel after towel drying | 3.3 | 3.3 |
| Sheen after towel drying | 3 | 3.7 |
| straightness after drying | 3 | 4.7 |
| Manageability | 3 | 4.7 |
| Overall appearance | 3 | 4.7 |
| Overall preference | 0 of 3 | 3 of 3 |

TABLE 5B
No. of subjects = 4.

| | Activator 1B | Activator 1C |
|---|---|---|
| Application | 4.75 | 4 |
| Breakdown | 4.25 | 4 |
| Rinseability | 4 | 4 |
| Odor | 4 | 4 |
| Irritation | 1 | 1 |
| Straightness after towel | 4.5 | 3.5 |
| drying | | |
| Feel after towel drying | 4 | 3 |
| Sheen after towel drying | 4 | 3 |
| straightness after drying | 4.25 | 3.25 |
| Manageability | 4.25 | 3.25 |
| Overall appearance | 4.5 | 3.5 |
| Overall preference | 4 of 4 | 0 of 4 |

TABLE 5C
No. of subjects = 3.

| | Activator 1C | Activator 1D |
|---|---|---|
| Application | 4 | 4.7 |
| Breakdown | 4 | 4.3 |
| Rinseability | 4.7 | 4 |
| Odor | 4.7 | 4.7 |
| Irritation | 1 | 1 |
| Straightness after towel | 4.3 | 4.7 |
| drying | | |
| Feel after towel drying | 3.7 | 2.7 |
| Sheen after towel drying | 3.7 | 3.3 |
| straightness after drying | 4.3 | 4.3 |
| Manageability | 4.3 | 3.7 |
| Overall appearance | 4.3 | 4 |
| Overall preference | 2 of 3 | 1 of 3 |

TABLE 5D
No. of subjects = 3.

| | Activator 1C | Activator 1E |
|---|---|---|
| Application | 4.7 | 4 |
| Breakdown | 4.7 | 4 |
| Rinseability | 3.7 | 4 |
| Odor | 4 | 4 |
| Irritation | 1 | 1 |
| Straightness after towel | 4.3 | 4 |
| drying | | |
| Feel after towel drying | 3.7 | 3.3 |
| Sheen after towel drying | 4 | 3.7 |
| straightness after drying | 4.3 | 4 |
| Manageability | 4.3 | 3.7 |
| Overall appearance | 4.3 | 3.7 |
| Overall preference | 2 of 3 | 1 of 3 |

TABLE 5E
No. of subjects = 3.

| | Activator 1C | Activator 1H |
|---|---|---|
| Application | 4.7 | 4 |
| Breakdown | 4.3 | 4 |
| Rinseability | 3.7 | 4 |
| Odor | 5 | 5 |
| Irritation | 1 | 1 |
| Straightness after towel | 4.3 | 4.3 |
| drying | | |
| Feel after towel drying | 3.7 | 3.3 |
| Sheen after towel drying | 4 | 3 |
| straightness after drying | 4 | 4.3 |
| Manageability | 4.3 | 3.3 |
| Overall appearance | 4.3 | 3.7 |
| Overall preference | 3 of 3 | 0 of 3 |

TABLE 5F
No. of subjects = 2.

| | Activator 1C | Activator 1F |
|---|---|---|
| Application | 5 | 4 |
| Breakdown | 4 | 4 |
| Rinseability | 4.5 | 4 |
| Odor | 4.5 | 4.5 |
| Irritation | 2 | 2 |
| Straightness after towel | 4.5 | 4.5 |
| drying | | |
| Feel after towel drying | 3.5 | 3.5 |
| Sheen after towel drying | 4 | 4 |
| straightness after drying | 4.5 | 4.5 |
| Manageability | 4 | 4 |
| Overall appearance | 4 | 4 |
| Overall preference | 1 of 2 | 1 of 2 |

TABLE 5G
No. of subjects = 3.

| | Activator 1C | Activator 1G |
|---|---|---|
| Application | 4.7 | 4 |
| Breakdown | 4.7 | 4.7 |
| Rinseability | 3.7 | 4 |
| Odor | 5 | 5 |
| Irritation | 1 | 1 |
| Straightness after towel | 5 | 4.7 |
| drying | | |
| Feel after towel drying | 3.7 | 3.3 |
| Sheen after towel drying | 3.7 | 3.3 |
| straightness after drying | 4.7 | 4.3 |
| Manageability | 4.3 | 3.7 |
| Overall appearance | 4.3 | 4 |
| Overall preference | 2 of 3 | 1 of 3 |

The results of Table 5A show that in this study, the Activators 1C and 2C performed comparably overall. Table 5B shows that the addition of Meadowquat® (3%) in Activator 1B resulted in a comparable or slightly improved performance as compared to Activator 1C. Based on the comments of the stylists, Activator 1C showed a substantial improvement over Activator 2C in terms of ease of mixing with Relaxer Base 3B.

### Example 6

This example demonstrates the overall performance and properties achieved by an exemplary relaxer gel activator of the invention relative to commercial products.

Activator 1B was compared to commercially available activators in the following hair straightening systems: Dark & Lovely (Activator 2D), Dr. Miracle's “Feel It” Formula Thermaceutical Intensive no Lye Relaxer System (Activator 5A), Organic Root Stimulator Olive Oil No Lye Relaxer System (Activator 5B), and Vitale Olive Oil Sensitive Scalp No Lye Relaxer (Activator 5C).

Activator 5A contained water, guanidine carbonate, glycerine, and xanthan gum. Activator 5B contained water, guanidine carbonate, sorbitol, and yellow #5. Activator 5C contained water, guanidine carbonate, sorbitol, Tetrasodium EDTA, olive extract, coconut extract, yellow #5, and blue #1.

The activators were tested on 24 test subjects using half-head evaluation with Activator 1B on one half and another activator (Activator 2D, 5A, 5B, or 5C) on the other half. For Activators 2D, 5A, 5B, and 5C, the hair straightening systems were used according to the manufacturer's instructions. The procedure for Activator 5A included a pre-treatment for previously relaxed hair. All of the systems included a relaxer step, a neutralizing shampoo step, and a post-relaxer conditioner step. When mixing the activator with the relaxer base, 213 g of the relaxer base was used and 60 g of the activator was used.

The procedure used for Activator 1B was as follows. Any previously relaxed hair was pre-treated with the Pre-Treatment Conditioner 3A of Example 3. Relaxer Base 3D (213 g) was mixed with Activator 1B (60 g) until homogeneous. The mixture was applied to the hair (not scalp) that had not been previously relaxed (the new growth). The total application and processing time did not exceed 15 minutes. Application timing was between 6-7 minutes, and processing timing was between 8-9 minutes. During the processing time, the new growth was smoothed into the straight position using either gloved fingers or the back of a comb. Two smoothing sessions were used. Caution was taken to prevent the mixture from contacting previously relaxed hair. This smoothing technique was used to straighten the hair out as much as possible. After 15 minutes, the hair was rinsed thoroughly (at least 5 minutes) with luke-warm water until there were no visible traces of the mixture left on hair. The Post Neutralizing Conditioner of Example 3 was applied to the hair and left on for 5 minutes. The hair was then rinsed thoroughly. The hair was then shampooed using a shampoo of Example 3, allowed to remain on the hair for 4-5 minutes, and rinsed. The shampoo step was repeated. The hair was then gently towel dried. The Masque Conditioner 3A of Example 3 was applied, allowed to remain on the hair for 5 minutes, and rinsed thoroughly.

The results of Tables 6A-6D were determined by the stylist (input from the subject was taken for irritation) on a 100% scale, where 0% was the least favorable and 100% was the most favorable.

The results comparing the applications of the activators are given in Table 6A.

TABLE 6A

| | Activator | Activator | Activator | Activator | Activator |
|---|---|---|---|---|---|
| | 1B | 2D | 5A | 5B | 5C |
| Ease of | 90 | 60 | 70 | 70 | 80 |
| Mixing | | | | | |
| Product | 90 | 70 | 70 | 70 | 75 |
| Consistency | | | | | |
| Product | 10 | 20 | 20 | 20 | 10 |
| Fall Off | | | | | |
| Ease of Pull | 90 | 70 | 75 | 75 | 80 |
| Through | | | | | |
| Break | 80 | 75 | 75 | 80 | 80 |
| Down of | | | | | |
| Natural Curl | | | | | |
| Ease of | 80 | 75 | 80 | 75 | 80 |
| Rinsing | | | | | |
| Scalp | 10 | 20 | 10 | 20 | 10 |
| Sensation | | | | | |

The wet hair results after neutralization are compared in Table 6B.

TABLE 6B

| | Activator | Activator | Activator | Activator | Activator |
|---|---|---|---|---|---|
| | 1B | 2D | 5A | 5B | 5C |
| Straightness | 80 | 75 | 80 | 75 | 80 |
| Smoothness | 90 | 70 | 75 | 75 | 80 |
| of Wet Hair | | | | | |
| Coating | 60 | 75 | 70 | 75 | 70 |
| (amount) | | | | | |
| wet hair | | | | | |
| Irritation | 20 | 75 | 50 | 75 | 35 |

The wet hair results after Masque conditioning are compared in Table 6C.

TABLE 6C

| | Activator | Activator | Activator | Activator | Activator |
|---|---|---|---|---|---|
| | 1B | 2D | 5A | 5B | 5C |
| Combing | 85 | 65 | 75 | 70 | 75 |
| Straightness | 80 | 70 | 80 | 75 | 80 |
| Smoothness | 80 | 70 | 80 | 80 | 80 |
| of Wet Hair | | | | | |
| Coating | 40 | 65 | 75 | 65 | 50 |
| (amount) | | | | | |
| wet hair | | | | | |
| Irritation | 10 | 40 | 30 | 30 | 25 |

The final dry hair observations are given in Table 6D.

TABLE 6D

| | Activator | Activator | Activator | Activator | Activator |
|---|---|---|---|---|---|
| | 1B | 2D | 5A | 5B | 5C |
| Straightness | 85 | 75 | 85 | 80 | 85 |
| Body | 85 | 65 | 80 | 70 | 80 |
| Shedding/ | 0 | 0 | 0 | 0 | 0 |
| Breakage | | | | | |
| Discipline | 80 | 65 | 70 | 65 | 70 |
| Shine | 85 | 70 | 75 | 70 | 75 |
| Smoothness | 85 | 70 | 75 | 70 | 80 |
| (Visual) | | | | | |
| Coating | 30 | 45 | 45 | 45 | 45 |
| (Amount) | | | | | |
| dry hair | | | | | |
| Final | 80 | 75 | 80 | 75 | 80 |
| Condition - | | | | | |
| Scalp | | | | | |

The Activator 1B showed the greatest ease of mixing and product consistency as well as ease of pull through (Table 6A). Activator 1B performed well in breakdown of natural curl, ease of rinsing, and reduced product fall off (Table 6A). After neutralization with the neutralizing shampoo, the Activator 1B system performed well in straightness (Table 6B). The wet hair data after the conditioning step also showed that the Activator 1B system performed well in straightness (Table 6C). Finally, Table 6D also shows that the Activator 1B system performed well in straightness. These results show not only that the relaxer system produced from Activator 1B performs comparably to commercial products in terms of straightening, but also show that Activator 1B exhibits unexpectedly superior mixing properties relative to commercial activators.

### Example 7

This example demonstrates the improvement in ease of mixing of an exemplary relaxer gel activator of the invention relative to a conventional liquid activator, and the improvement in overall appearance and uniformity of the relaxer composition achieved thereby.

Two no lye relaxer compositions were prepared, Comparative Sample A and Sample B. Comparative Sample A was prepared from a conventional liquid activator and Sample B was prepared from a relaxer gel activator of the present invention.

To each of two 400 ml Pyrex beakers, A and B, 213 g of no lye Relaxer Base 3D (prepared as described in Example 3) was added. 60 g of a comparative liquid Activator 2A (prepared as described in Example 2) was added to beaker A. 60 g of an exemplary gel Activator 1B (prepared as described in Example 1) was added to beaker B. Both beakers were separately attached with a chain clamp under an IKA Eurostar Power Basic Stirrer equipped with a flat 2.5 inch metal mixing blade. The mixtures in beakers A and B (containing Comparative Sample A and Sample B, respectively) were mixed for 3 minutes at 300 rpm/min speed.

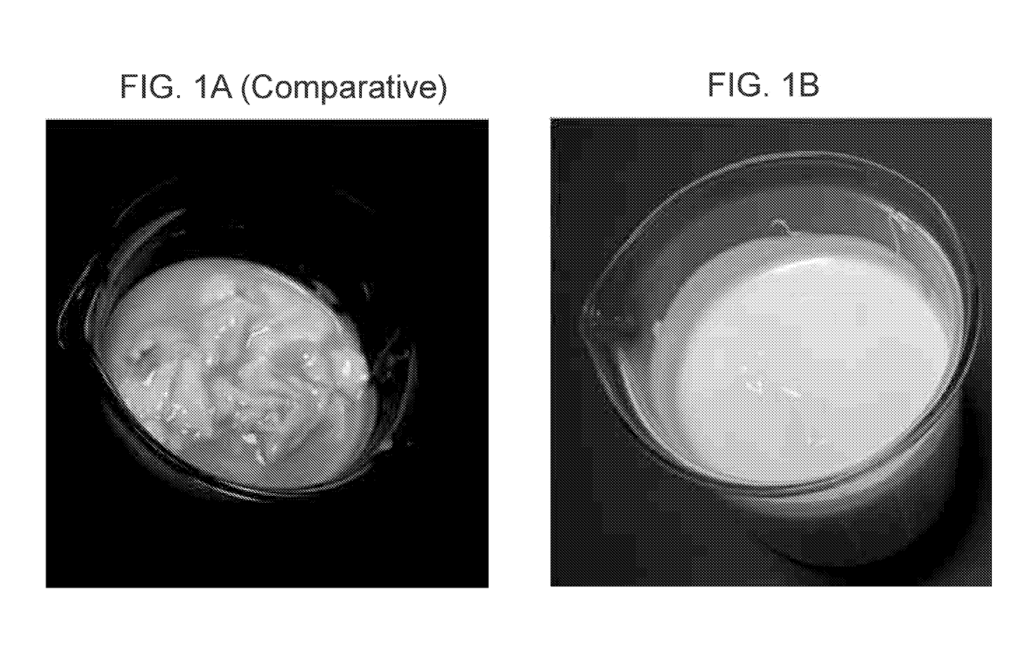

Comparative Sample A, after mixing, produced a clumpy mixture, which appeared to contain at least two distinct phases, one that was thick and interspersed with another phase that had a watery consistency (FIG. 1A). After mixing, Sample B produced a creamy, homogeneous mixture (FIG. 1B). The foregoing results show that the relaxer gel activator of the present invention exhibited superior mixing properties and produced a relaxer composition that was significantly more uniform than the relaxer composition produced from the conventional liquid activator. The gel activator of the invention produced a relaxer composition with improved uniformity and consistency over the comparative relaxer composition, which one of ordinary skill in the art will appreciate will result in a substantial improvement in application and use.

### Example 8

This example demonstrates an exemplary kit comprising compositions of the invention.

A hair relaxer kit (Silkening Shine No-Lye Relaxer System) is prepared as follows. A Conditioning Gel Activator is added to a cylindrical plastic container, providing a net weight of 1.8 ounces (51 grams) of relaxer gel activator, and the container is sealed with a child-resistant screw-top cap. The activator is either Regular strength (Activator 11 of Example 3, 28% guanidine carbonate) or Super strength (Activator 1J of Example 3, 28.2% guanidine carbonate). No Lye Crème Relaxer (Relaxer Base 3D of Example 3) is added to a slightly beveled cylindrical plastic container, providing a net weight of 7.5 ounces (212 grams) of relaxer base, and the container is topped with a plastic lid, where the lid is protectively sealed to the container using clear plastic. Pre-Treatment Conditioner (Pre-Treatment Conditioner 3B of Example 3) is added to a rippable plastic pouch, providing 1.5 fluid ounces (44.3 ml). Neutralizing Shampoo with Color Code (Neutralizing Shampoo 3B of Example 3), Silkening Conditioning Masque (Masque conditioner 3B of Example 3), and Leave-In Conditioner (Leave-In Conditioner of Example 3) are each added to separate cylindrical plastic containers, each providing 1.8 fluid ounces (53 ml), and the containers are capped with screw caps.

The containers of the above compositions are appropriately labeled prior to addition of the compositions. After the compositions are added to the containers, the containers are added to an appropriately labeled box that identifies the compositions and ingredients of the compositions. A pair of latex gloves are placed in a resealable plastic bag which is then placed in the box. A wooden spatula is also added to the box.

Instructions are added to the box. The instructions are for a home user to effectively use the kit to straighten hair at home. The instructions list the contents of the kit as those items given above. The instructions also state additional items are needed that are not included in the kit. These items include a timer, a wide-tooth comb, and petroleum jelly or a scalp base. The instructions further state that it is important to protect the hairline and ears with petroleum jelly or scalp base and that these areas are the most delicate and can become easily irritated; if the user has a sensitive scalp, the user should make sure to part the hair and apply the petroleum jelly or scalp base directly to the entire scalp (not the hair) before applying the relaxer; and to review all the warnings and do's and don'ts listed therein.

The instructions provide warnings regarding the relaxer compositions of the kit and against misuse of the kit. The warnings are substantially as follows: Keep out of the reach of children. This product contains an alkali agent and guanidine carbonate (toxic). Keep this product and all others away from eyes and mouth. Avoid contact with eyes, skin, scalp, and mouth. Product may cause blindness. If product gets in eyes, rinse thoroughly with water. If product is swallowed or eaten do not induce vomiting, consult a physician or Poison Control Center. Contact with skin, or scalp may cause severe injury. Misuse of this product can cause hair and skin damage. Follow directions carefully to avoid skin and scalp burns, hair loss and eye injury. You must use suitable gloves when applying and rinsing product. Do not use on bleached or permanently colored hair. Do not use on hair that is breaking, splitting, shedding, or damaged. If hair has been previously processed, apply only to the new growth as described in the directions. Apply protective gel around entire hairline, neck, ears and forehead, before applying product. If product causes skin or scalp irritations, rinse out immediately and wash with the shampoo in the kit. If skin or scalp irritation persists, consult a physician. This product should be applied by an adult. Discard any mixed excess portion of product.

The instructions also provide “Do's and Don'ts” substantially as follows:

Do follow directions carefully.

Do keep out of reach of children—some products may be toxic if ingested.

Do consult a physician if product is accidentally eaten or swallowed. If product is swallowed or eaten do not induce vomiting, consult a physician or Poison Control Center.

Do avoid product contact with eyes. Rinse out immediately with water and consult a physician.

Do keep activator jar closed at all times.

Do keep hair away from sparks and open flames at all times.

Do check scalp carefully for irritations, cuts, and/or abrasions. If present, don't use product until scalp condition clears up.

Do conduct an allergy and strand test prior to use.

Do apply petroleum jelly around entire hairline, neck, ears and forehead, before applying product.

Do use gloves when applying and rinsing product.

Do use a clock or timer for accurate timing of chemicals on hair.

Do apply product to new growth as described in the directions.

Do remove product from skin (ears, neck or face) by wiping gently with a damp cloth.

Do rinse immediately if scalp irritation occurs. If skin and scalp irritation persist, consult a physician.

Do discard any excess mixed product immediately after use.

Don't use on bleached or permanently colored hair.

Don't use on hair which is breaking, splitting, shedding, or damaged.

Don't use product if you have used an ammonia thioglycolate product (curly perm). Wait at least six months until new hair grows out.

Don't shampoo hair at least 48 hours prior to using product.

Don't scratch or brush scalp prior to use of product.

Don't cut hair with razors or clippers immediately prior to application process.

Don't pull, comb, or stretch hair during application.

Don't use heat in any step of the process except styling.

Don't repeat application process for at least 4-6 weeks.

The instructions list preliminary steps to take prior to applying relaxer to the full head of hair. These steps include an allergy test and a strand test which are substantially as follows:

Before you start the relaxing process make sure you do an allergy test followed by a strand test.

Allergy Test (prior to strand test)

1. Mix No-Lye Crème Relaxer and Conditioning Gel Activator according to instructions (see section titled, “How to Mix” below).

2. Apply a small amount of No-Lye Crème Relaxer mixture on the inner arm, near the elbow, and leave on 2-3 minutes. Do not exceed 3 minutes.

3. Remove the relaxer by rinsing the area thoroughly with lukewarm water.

4. Examine area. If allergic reaction (such as redness or rash) appears, do not proceed.

Strand Test

The strand test determines proper amount of time for relaxing hair your hair.

1. Check scalp carefully. If cuts or abrasions are present do not use relaxer until scalp condition clears. Use the mixture prepared for the Allergy Test (See section titled, “How to Mix” below).

2. Separate several strands of hair near the nape of the neck and apply a small amount of the relaxer to the unrelaxed hair.

3. Leave relaxer on for recommended time (See Time Chart below, Table 7). Apply a small amount of Neutralizing Shampoo with Color Code only to the relaxer-tested hair and rinse thoroughly (important: only shampoo the strands that have been tested).

4. Examine the hair strands. If test shows breakage or damage, do not use this product until hair is in better condition. Regular use of a conditioner will help improve the condition of the hair.

The instructions continued with the “How to Mix” section. The instructions were substantially as follows:

Do not mix the products until you are ready to use.

Remove lid from plastic jar containing Silkening Shine Relaxer Base (the No-Lye Crème Relaxer Base). Use enclosed wooden spatula to remove all of Conditioning Gel Activator contents and transfer into the Relaxer Base. Important: The Relaxer base and Conditioning Gel Activator desirably should be completely blended together for the best results. Mix until the combination is free of lumps and is a creamy pale pink color (about 1 minute). Note: If there are streaks of pink, continue to mix until the mixture is one solid color. Do not allow mixture to stand for more than 30 minutes before using. Replace the lid and immediately discard any unused product after relaxing.

For best results, follow all instructions carefully.

Check timing guide before starting. Make sure everything you need is close at hand. Apply petroleum jelly or scalp base to entire hairline, nape of the neck and ears. Gently comb through hair to untangle and section into four parts by creating a part from front to back and another part from ear to ear. Once you have four equal sections, cover your shoulders with a towel. Put on latex gloves and set the timer for the maximum time for your hair type as indicated on the Time Chart (Table 7 below) and Strand Test (above).

The instructions then gave step-by-step instructions for application of the relaxer mixture, the instructions being substantially as follows:

1. Application (Virgin Hair)

Apply petroleum jelly or scalp base to the hairline, nape of neck and ear area prior to applying the relaxer. Since the hair has not been previously relaxed, there is no need to apply the Pre-Treatment Conditioner.

Wear gloves during application.

Beginning with the back section, part hair into one-fourth (¼) sections. Apply a generous amount of Silkening Shine Crème Relaxer mixture with the back of a wide tooth comb, about one-half (½) inch away from the scalp to one-half (½) inch away from the ends. Continue moving through until the section is complete, before moving to the next section. Discard any excess, mixed product immediately after application is fully completed.

2. Application—Retouch (Previously relaxed hair)

Apply the Pre-Treatment Conditioner to all previously relaxed hair. The Pre-Treatment Conditioner helps protect previously relaxed hair from becoming over-dried and brittle. Avoid applying Pre-Treatment Conditioner on the new growth hair.

Apply to new growth hair only. Starting at the back section, apply a generous amount of Silkening Shine Crème Relaxer to one-fourth (¼) sections. However, be sure not to overlap relaxer on previously relaxed hair. Be sure to wait at least every 4 to 6 weeks for relaxer retouching. Discard any excess mixed product immediately after application is fully completed.

3. Smoothing

When relaxer application is finished, begin smoothing where the relaxer was first applied. Use the back of the comb or fingers to smooth the relaxer through the hair. Use firm strokes. On virgin hair, smooth from scalp to ends. On retouches, limit smoothing to the new growth only. Do not exceed recommended processing time. Discard gloves after use.

4. Rinse

Thoroughly rinse the hair with warm water to remove all of the relaxer. Keep your eyes tightly closed and keep relaxer away from your face. While rinsing, use your fingers to gently open parts of the hair so the water can work through the hair and remove relaxer. Pay special attention to the ears, nape of neck and hairline areas.

5. Shampoo hair with Neutralizing Shampoo with Color Code

Apply a generous amount of Neutralizing Shampoo with Color Code to hair and work into lather. This shampoo will indicate where the Crème Relaxer remains in the hair by producing a pink lather. Rinse well and repeat process until all relaxer is rinsed out of hair. When all the Crème Relaxer is removed from the hair and around the ears, forehead and nape of neck, the lather will be white. Rinse thoroughly for at least 2 to 4 minutes.

6. Apply Silkening Conditioning Masque

When hair has been thoroughly rinsed of shampoo lather, apply a generous amount of Silkening Conditioning Masque over the entire head. Thoroughly massage masque in from the scalp to the ends. Make sure all hair is completely saturated with the masque. Leave on for 5-7 minutes, then rinse thoroughly with lukewarm water. Blot dry with a towel.

Silkening Conditioning Masque incorporates a nourishing blend of conditioning agents to thoroughly moisturize your hair. Silk extracts and silicones restore hair's pH levels and fortify hair against breakage. Leaves hair strong, supple and super lustrous.

7. Apply Leave-In Conditioner

Apply Leave-In Conditioner and gently distribute evenly throughout the hair. Do not rinse. Leave-In Conditioner's conditioning humectants give hair an extra moisture boost and add a protective shield against damage caused by heat styling.

Towel blot excess and style as desired.

The Time Charts are given in Table 7

TABLE 7

| Time Chart for Regular Strength | | Time Chart for Super Strength | |
|---|---|---|---|
| Hair Texture | Maximum Time | Hair Texture | Maximum Time |
| Color | Do not use | Color | Do not use |
| Treated/Bleached | | Treated/ | |
| | | Bleached | |
| Fine | 10-12 minutes | Fine | Do not use |
| Medium | 12-15 minutes | Medium | Do not use |
| Coarse/Medium | 15-20 minutes | Coarse | 18-20 minutes |
| Coarse | | | |

### Example 9

This example illustrates exemplary compositions of the invention.

Pre-treatment conditioner: water (aqua, eua), cetearyl alcohol, PEG-12 dimethicone, sorbitan oleate, hydroxypropyltrimonium honey, glycerin, cocamide MEA, polyquaternium-32, oleth-5, oleth-20, cocos nucifera (coconut) oil, ceteareth-20, polyquaternium-22, behentrimonium chloride, phenoxyethanol, mineral oil (paraffinum liquidum, huile minerale), hydroxyethylcellulose, fragrance (parfum), methylparaben, isopropyl alcohol, PPG-1 trideceth-6, butyrospermum parkii (shea butter), aloe barbadensis leaf juice, disodium EDTA, hexyl cinnamal, limonene, amyl cinnamal, linalool.

No lye relaxer crème: water (aqua, eua), petrolatum, mineral oil (paraffinum liquidum, huile minerale), calcium hydroxide, propylene glycol, stearyl alcohol, cetyl alcohol, ceteth-20, steareth-20, fragrance (parfum), hexyl cinnamal, limonene, amyl cinnamal, linalool.

Conditioning gel activator: water (aqua, eua), guanidine carbonate, PEG-2 dimeadowfoamamidoethylmonium methosulfate, glycerin, carbomer, hexylene glycol, aloe barbadensis leaf juice, red 33 (CI 17200).

Neutralizing shampoo with color code: water (aqua, eua), sodium laureth sulfate, cocamidopropyl betaine, PEG-30 glyceryl cocoate, PPG-5-ceteth-10 phosphate, fragrance (parfum), sodium C14-C17 alkyl sec sulfonate, disodium cocoamphodipropionate, polyquaternium-10, PEG-12 dimethicone, PEG-120 methyl glucose dioleate, citric acid, cocotrimonium chloride, DMDM hydantoin, hexyl cinnamal, limonene, amyl cinnamal, linalool, phenolsulfonphthalein.

Silkening conditioning masque: water (aqua, eua), stearyl alcohol, cyclopentasiloxane, cetyl alcohol, stearamidopropyl dimethylamine, dimethicone, cyclohexasiloxane, aspartic acid, quaternium-18, fragrance (parfum), DMDM hydantoin, isostearamidopropyl ethyldimonium ethosulfate, theobroma cacao (cocoa) seed butter, honey (mel, miel), cocos nucifera (coconut) oil, citric acid, silk amino acids, hexyl cinammal, PEG-9, disodium EDTA, limonene, amyl cinnamal, linalool, panthenol, propylene glycol, polyglyceryl-3 distearate, polysorbate 60, chamomilla recutita (matricaria) flower extract, stearic acid, palmitic acid, myristic acid, guar hydroxypropyltrimonium chloride, triticum vulgare (wheat) flour lipids, persea gratissima (avocado) oil, tetrahexyldecyl ascorbate, glycine soja (soybean) sterols, alpha-glucan oligosacchride, ceramide-3, ethylhexyl methoxycinnamate, butyl methoxydibenzoylmethane, tococpheryl acetate.

Leave-in conditioner: water (aqua, eua), amodimethicone, glycerin, propylene glycol, PEG-12 dimethicone, cetrimonium chloride, PVP, polysorbate 20, fragrance (parfum), DMDM hydantoin, benzophenone-4, trideceth-12, disodium EDTA, triethanolamine, hexyl cinnamal, limonene, amyl cinnamal, linalool, cocos nucifera (coconut) oil, polyglyceryl-3 distearate, polysorbate 60, guar hydroxypropyltrimonium chloride, myristic acid, palmitic acid, stearic acid, honey extract (mel, extrait de miel), triticum vulgare (wheat) flour lipids, persea gratissima (avocado) oil, tetrahexyldecyl ascorbate, glycine soja (soybean) sterols, alpha-glucan oligosacchride, ceramide-3, ethylhexyl methoxycinnamate, butyl methoxydibenzoylmethane, tocopheryl acetate.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A gel composition for relaxing hair fiber, the composition comprising:
a carbomer;
an alkaline neutralizing agent consisting essentially of guanidine carbonate; and
a fluid medium;
wherein the carbomer is present in an amount effective to form a gel in the fluid medium and the guanidine carbonate is present in an amount effective to neutralize the carbomer sufficiently to produce a gel in the fluid medium.

2. A gel composition for relaxing and/or straightening hair fiber, the composition comprising:
a carbomer;
a neutralizing agent comprising guanidine carbonate; and
a fluid medium;
wherein the gel composition is substantially free of any alkaline neutralizing agent other than guanidine carbonate, the carbomer is present in an amount effective to form a gel in the fluid medium, and the guanidine carbonate is present in an amount effective to neutralize the carbomer sufficiently to produce a gel in the fluid medium.

3. The composition of claim 1, wherein the carbomer is present in an amount of from about 1 wt. % to about 5 wt. % of the composition.

4. The composition of claim 1, wherein the guanidine carbonate is present in an amount of from about 25 wt. % to about 35 wt. % of the composition.

5. The composition of claim 1, further comprising at least one humectant, at least one color indicator, at least one conditioning agent, or any combination thereof.

6. The composition of claim 5, wherein the at least one conditioning agent is PEG-2 dimeadowfoamamidoethylmonium methosulfate.

7. The composition of claim 5, wherein the at least one humectant is glycerin, or propylene glycol or a combination thereof.

8. The composition of claim 1, wherein the fluid medium comprises water.

9. The composition of claim 1, further comprising PEG-2 dimeadowfoamamidoethylmonium methosulfate, and glycerin, wherein the fluid medium comprises water.

10. A method for relaxing hair fiber, the method comprising:
reacting the gel composition of claim 1 with a hydroxide composition to form guanidine hydroxide; and
contacting the hair fiber with the guanidine hydroxide in an amount effective to relax and/or straighten the hair fiber,
wherein the hydroxide composition comprises a carrier and a hydroxide compound in an amount effective to convert at least a portion of the guanidine carbonate into guanidine hydroxide when mixed with the gel composition.

11. The method of claim 10, wherein the hydroxide composition further comprises at least one conditioning compound.

12. The method of claim 11, wherein the conditioning compound comprises an alkyl quaternary ammonium salt.

13. The method of claim 10, wherein the hydroxide composition is a cream comprising calcium hydroxide in an amount effective to produce the guanidine hydroxide.

14. The method of claim 10, wherein the hydroxide composition further comprises at least one long-chain hydrocarbon, at least one fatty alcohol, at least one humectant, at least one fatty-ethylene oxide, or at least one preservative, or any combination thereof.

15. The method of claim 14, wherein the at least one long-chain hydrocarbon is petrolatum and/or mineral oil, the at least one fatty alcohol is stearyl alcohol and/or cetyl alcohol, the at least one humectant is glycerin and/or propylene glycol, the at least one fatty-ethylene oxide is steareth-20 and/or ceteth-20, and/or the at least one preservative is benzyl benzoate.

16. The method of claim 15, wherein the hydroxide composition further comprises hexyl cinnamal, linalool, limonene, or amyl cinnamal, or a combination thereof.

17. The method of claim 10, wherein the hydroxide composition further comprises
petrolatum, mineral oil, propylene glycol, cetyl alcohol, stearyl alcohol, ceteth-20, and steareth-20,
wherein the carrier is water.

18. The method of claim 10, further comprising allowing the guanidine hydroxide to maintain contact with the hair fibers for about 10-20 minutes.

19. The method of claim 10, further comprising mechanically straightening the hair fiber.

20. The method of claim 10, further comprising rinsing the hair fiber after contacting with the guanidine hydroxide.

21. A method for producing a gel composition for relaxing and/or straightening hair fiber, the method comprising neutralizing carbomer with guanidine carbonate in a fluid medium to produce the gel composition, wherein the gel composition is substantially free of any alkaline neutralizing agent other than guanidine carbonate.

22. A kit comprising:
(a) a first container and the gel composition of claim 1 contained therein; and
(b) a second container and a hydroxide composition contained therein.

23. The kit of claim 22, further comprising instructions (i) for reacting the gel and hydroxide compositions to produce guanidine hydroxide, and (ii) for contacting hair with the guanidine hydroxide to relax and/or straighten the hair.

24. The kit of claim 22, wherein the hydroxide composition further comprises at least one conditioning compound.

25. The kit of claim 24, wherein the conditioning compound comprises an alkyl quaternary ammonium salt.

26. The kit of claim 22, wherein the hydroxide composition is a cream comprising calcium hydroxide in an amount effective to produce the guanidine hydroxide.

27. The kit of claim 22, wherein the hydroxide composition further comprises at least one long-chain hydrocarbon, at least one fatty alcohol, at least one humectant, at least one fatty-ethylene oxide, or at least one preservative, or any combination thereof.

28. The kit of claim 22, wherein the hydroxide composition further comprises at least one long-chain hydrocarbon, at least one fatty alcohol, at least one humectant, at least one fatty-ethylene oxide, or at least one preservative, or any combination thereof.

29. The kit of claim 28, wherein the at least one long-chain hydrocarbon is petrolatum and/or mineral oil, the at least one fatty alcohol is stearyl alcohol and/or cetyl alcohol, the at least one humectant is glycerin and/or propylene glycol, the at least one fatty-ethylene oxide is steareth-20 and/or ceteth-20, and/or the at least one preservative is benzyl benzoate.

30. The kit of claim 29, wherein the hydroxide composition further comprises hexyl cinnamal, linalool, limonene, or amyl cinnamal, or a combination thereof.

31. The kit of claim 22, wherein the hydroxide composition further comprises
petrolatum, mineral oil, propylene glycol, cetyl alcohol, stearyl alcohol, ceteth-20, and steareth-20,
wherein the carrier is water.

32. The composition of claim 2, wherein the carbomer is present in an amount of from about 1 wt. % to about 5 wt. % of the composition.

33. The composition of any claim 2, wherein the guanidine carbonate is present in an amount of from about 25 wt. % to about 35 wt. % of the composition.

34. The composition of claim 2, further comprising at least one humectant, at least one color indicator, at least one conditioning agent, or any combination thereof.

35. The composition of claim 34, wherein the at least one conditioning agent is PEG-2 dimeadowfoamamidoethylmonium methosulfate.

36. The composition of claim 34, wherein the at least one humectant is glycerin, propylene glycol or a combination thereof.

37. The composition of claim 2, wherein the fluid medium comprises water.

38. The composition of claim 2, further comprising PEG-2 dimeadowfoamamidoethylmonium methosulfate and glycerin, wherein the fluid medium comprises water.
